(19) United States
(12) Patent Application Publication
(10) Pub. No.: US 2011/0092601 A1
(43) Pub. Date: Apr. 21, 2011

(54) LYSINE-SPECIFIC DEMETHYLASE INHIBITORS

(75) Inventors: Patrick M. Woster, Canton, MI (US); Robert A. Casero, Glen Arm, MD (US)

(73) Assignee: THE JOHNS HOPKINS UNIVERSITY, Baltimore, MD (US)

(21) Appl. No.: 12/595,955

(22) PCT Filed: Apr. 14, 2008
(86) PCT No.: PCT/US2008/004874

§ 371 Date: Jan. 4, 2011

Related U.S. Application Data
(60) Provisional application No. 60/911,692, filed on Apr. 13, 2007.

(51) Int. Cl. A61K 31/155; C07C 279/12; C07C 211/01; C07C 251/00; A61P 35/00

(52) U.S. Cl. 514/634; 564/236; 564/233; 564/512; 564/461; 564/509; 560/168; 562/560; 564/159; 564/197; 560/124; 562/506; 564/152; 564/192

(57) ABSTRACT

Polyamine, polyamine/guanidino, and polyamine/biguanide compounds bearing allene, propargyl, cyclopropyl, and other reactive moieties are disclosed. The compounds are useful as irreversible inhibitors of the enzyme lysine-specific demethylase-1 and for the treatment of cancer.

The present application claims the benefit of U.S. provisional application No. 60/911,692 filed Apr. 13, 2007, which is incorporated herein by reference in its entirety.

### TECHNICAL FIELD

This invention pertains to polyamine compounds, polyamine/guanidine compounds, and polyamine/biguanide compounds, which bear allene, propargyl, alkynyl, cyclopropyl, choromethyl ketone, or other reactive moieties and which are useful for inhibition of lysine-specific demethylase. The compounds are useful in treatment of cancer.

### BACKGROUND

Polyamines are found in both eukaryotic and prokaryotic cells and figure prominently in regulation of the cell cycle and cell division. Agents specifically targeting polyamine biosynthesis, such as polyamine analogs, have been shown to have therapeutic effect in treatment of cancer, parasitic diseases, and other indications. These antiproliferative effects have been demonstrated to be, in part, a result of agent-induced decreases in the natural intracellular polyamines resulting from inhibition, down-regulation of polyamine biosynthesis and/or up regulation of polyamine catabolism. See, e.g., Wang and Casero, J, Biochem. 139:17 (2006); Casero et al., Proc. West. Pharmacol. Soc. 48:24 (2005); Casero et al., J. Med. Chem. 44:1 (2001); U.S. Pat. Nos. 5,889,061, 6,392,098, and 6,794,545; U.S. Patent Application Publication Nos. 2003/0072715, 2003/0195377, and International Patent Applications Nos. WO 98/17624, WO 00/66587, WO 02/10142, and WO 03/050072. Bi et al., Bioorgan. Med. Chem. Letters 16:3229 (2006) discuss novel alkylpolyaminoguanidines and alkylpolyaminobiguanides with potent antitrypanosomal activity.

The recently discovered enzyme lysine-specific demethylase 1 (LSD1) has been shown to play a significant role in epigenetic control of gene expression (see Shi et al., Cell 119:941 (2004) and International Patent Application No. WO 2006/071608). The LSD1 enzyme appears to be up-regulated in some forms of human cancer (see Huang, Y.; Greene, E.; Murray-Stewart, T.; Goodwin, A. C.; Baylin, S. B.; Woster, P. M.; Casero, R. A.: Inhibition of the lysine specific demethylase, LSD1, by novel polyamine analogues results in re-expression of aberrantly silenced genes. Proc. Natl. Acad. Sci. U.S.A., 2007, not yet published). Dimethyl lysine 4 histone H3 (H3K4me2) is a transcription activating chromatin mark at gene promoters, and demethylation of this mark by LSD1, a homologue of polyamine oxidases, may broadly repress gene expression. As such, specific inhibitors for LSD1 have the potential to act as antitumor agents by limiting the demethylation of dimethyl lysine 4 histone H3 (H3K4me2), thus promoting the reexpression of multiple, aberrantly silenced genes. A novel series of polyaminoguanidines and polyaminobiguanides that are non-competitive inhibitors of LSD1 has been described (see International Patent Application No. WO 2007/0218392 and Bi et al., Bioorg. Med. Chem. Lett. 16:3229 (2006)), and that promote the reexpression of secreted frizzle-related proteins (SFRPs) and the GATA-family of transcription factors, which are important in the development of colon cancer. These events are concurrent with increased H3K4me2, decreased H3K9me1 and H3K9me2 repressive marks, and for some genes, reduced CpG island DNA methylation at the promoter of these genes. These agents provide an important new class of antitumor therapy.

The current application discloses LSD1 inhibitors that bear allene, propargyl, alkynyl, cyclopropyl, choromethyl ketone, or other moieties that will form covalent bonds in the LSD1 active site, and thus provides a novel set of irreversible inhibitors of LSD1 .

### DISCLOSURE OF THE INVENTION

The invention embraces polyamine, polyamine/guanidine, and polyamine/biguanide compounds having at least one functional group selected from —C₁-C₈ alkyl-CH═C═CH₂, —C₁-C₈ alkyl-C≡CH, —C₁-C₈ alkyl-cyclopropane, —C(═O)C₁-C₈ alkyl substituted with at least one halo group and -cyclopropyl-NR₂ where each R is independently H, alkyl, or aralkyl, and uses of those compounds for treatment and prevention of cancer. In one embodiment, the invention embraces polyamine, polyamine/guanidine, and polyamine/biguanide compounds having at least one functional group selected from —N(CH₃)(C₁-C₈ alkyl-CH═C═CH₂), —N(CH₃)(C₁-C₈ alkyl-C≡CH), —N(CH₃)(C₁-C₈ alkyl-cyclopropane), —N(CH₃)(C(═O)C₁-C₈ alkyl substituted with at least one halo group) and —C₁-C₈alkyl-cyclopropyl-NR₂ where each R is independently H, alkyl, or aralkyl, and uses of those compounds for treatment and prevention of cancer. In one embodiment, the invention embraces polyamine, polyamine/guanidine, and polyamine/biguanide compounds having at least one functional group selected from allene (—CH═C═CH₂), propargyl (—CH₂—C≡CH), cyclopropylmethyl

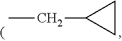

chloromethylcarbonyl(—C(═O)CH₂Cl) and 1-N-methylaminecycloprop-2-yl

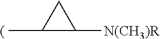

where R is C₁-C₈alkyl or aralkyl where the alkyl moiety of the aralkyl group is a C₁-C₈ alkyl), and uses of those compounds for treatment and prevention of cancer. In another embodiment, the invention embraces polyamine, polyamine/guanidine, and polyamine/biguanide compounds having at least one functional group selected from —N(CH₃)(CH₂—CH═C═CH₂), —N(CH₃)(CH₂—C≡CH), —N(CH₃)

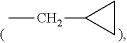

### (—N(CH₃)(C(═O)CH₂Cl) and —CH₂CH₂

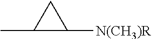

where R is C₁-C₈alkyl or aralkyl where the alkyl moiety of the aralkyl group is a C₁-C₈ alkyl), and uses of those compounds for treatment and prevention of cancer. In one embodiment, the compounds are derivable from lysine and have the functional groups —NR—CH(—COOH)—(CH₂)₄NR—, —NR—CH(—COOR)—(CH₂)₄NR—, or —NR—CH(—CONHR)—(CH₂)₄NR— where each R is independently H, C₁-C₈ alkyl or aralkyl where the alkyl moiety of the aralkyl group is a C₁-C₈ alkyl. The invention also embraces uses of those compounds for inhibition of lysine-specific demethylase-1, and treatment of diseases involving lysine-specific demethylase-1.

In one embodiment, the invention embraces compounds of the formula (M):

E-X-A-NH—B—NH-A-X-E (M)

where each E is independently selected from hydrogen, C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl or heteroaryl, C₇-C₂₄ substituted or unsubstituted aralkyl or heteroalkyl or heteroaralkyl, C₃-C₂₄ substituted or unsubstituted heteroaryl, or C₁-C₈ alkyl-CH═C═CH₂ or C₁-C₈ alkyl-C≡CH or C₁-C₈ alkyl-cyclopropane or C(═O)C₁-C₈alkyl substituted with at least one halo group or cyclopropyl-NR₂ where each R is independently H, alkyl, or aralkyl, with the proviso that at least one E is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; each A is independently a C₁-C₈ n-alkyl; B is independently selected from C₁-C₁₂ n-alkyl or C₃-C₈ cycloalkyl; and each X is independently selected from —NH—, —N(CH₃)—, —NH—C(═NH)—NH—, and —NH—C(═NH)—NH—C(═NH)—NH—; and all salts, solvates, hydrates, and stereoisomers thereof.

In another embodiment, B is independently selected from C₁-C₈ n-alkyl. In another embodiment, B is —(CH₂)₇—.

In another embodiment, at least one E is C₁-C₈ alkyl-CH═C═CH₂. In one embodiment, the at least one E that is C₁-C₈ alkyl-CH═C═CH₂ is C₁-C₈ n-alkyl-CH═C═CH₂. In another embodiment, the at least one E that is C₁-C₈ alkyl-CH═C═CH₂ is —CH₂—CH═C═CH₂.

In another embodiment, at least one E is C₁-C₈ alkyl-C≡CH. In one embodiment, the at least one E that is C₁-C₈ alkyl-C≡CH is C₁-C₈ n-alkyl-C≡CH. In another embodiment, the at least one E that is C₁-C₈ alkyl-C≡CH is propargyl (—CH₂—C≡CH).

In another embodiment, at least one E is C₁-C₈ alkyl-cyclopropane. In one embodiment, the at least one E that is C₁-C₈ alkyl-cyclopropane is C₁-C₈ n-alkyl-cyclopropane. In another variation, the at least one E that is C₁-C₈ alkyl-cyclopropane is cyclopropylmethyl

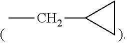

In another embodiment, at least one of E is C(═O)C₁-C₈alkyl substituted with at least one halo group. In one embodiment, the at least one of E that is C(═O)C₁-C₈alkyl substituted with at least one halo group is C(═O)C₁-C₈ n-alkyl substituted with at least one halo group selected from chloro or fluoro. In one embodiment, the at least one of E that is C(═O)C₁-C₈alkyl substituted with at least one halo group is C(═O)C₁-C₄ n-alkyl substituted with at least one chloro group. In another embodiment, the at least one of E that is C(═O)C₁-C₈alkyl substituted with at least one halo group is C(═O)(CH₂)ₙCH₂Cl where n is 1-7. In another embodiment, the at least one of E that is C(═O)C₁-C₈alkyl substituted with at least one halo group is chloromethylcarbonyl (C(═O)CH₂Cl).

In another embodiment, at least one of E is cyclopropyl-NR₂ where each R is independently H, alkyl, or aralkyl. In one embodiment, the at least one E that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety of the aralkyl group is a C₁-C₈ alkyl, which may be a C₁-C₈ n-alkyl. In another embodiment, the at least one E that is cyclopropyl-NR₂ where each R is independently H, alkyl, or aralkyl is 1-N-methylaminecycloprop-2-yl

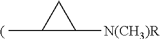

where R is C₁-C₈alkyl or aralkyl where the alkyl moiety of the aralkyl group is a C₁-C₈ alkyl). In another embodiment, the at least one E that is cyclopropyl-NR₂ where each R is independently H, alkyl, or aralkyl is 1-N,N-dimethylaminocycloprop-2-yl.

In another embodiment, at least one X is selected from —NH—C(═NH)—NH— and —NH—C(═NH)—NH—C(═NH)—NH—. In another embodiment, at least one X is —NH—C(═NH)—NH—. In another embodiment, at least one X is —NH—C(═NH)—NH—C(═NH)—NH—. In another embodiment, each X is independently selected from —NH—C(═NH)—NH— and —NH—C(═NH)—NH—C(═NH)—NH—. In another embodiment, both X groups are —NH—C(═NH)—NH—. In another embodiment, both X groups are —NH—C(═NH)—NH—C(═NH)—NH—. In another embodiment, one X is —NH—C(═NH)—NH— and another X is —NH—C(═NH)—NH—C(═NH)—NH—.

In one embodiment, the invention embraces polyamine/guanidine or N-alkylated polyamine/guanidine compounds having at least one functional group selected from —C₁-C₈ alkyl-CH═C═CH₂, —C₁-C₈ alkyl-C≡CH, —C₁-C₈ alkyl-cyclopropane, —C(═O)C₁-C₈ alkyl substituted with at least one halo group and -cyclopropyl-NR₂ where each R is independently H, alkyl, or aralkyl, such as a polyaminobisguanidine or polyaminobiguanide or N-alkylated variation thereof. An N-alkylated polyaminoguanidine intends a polyaminoguanidine wherein the imine nitrogen of the guanidine is alkylated, such as in a 2-methylguanadine derivative. In one embodiment, each A is —(CH₂)₃— and B is —(CH₂)₄—. In another embodiment, each A is —(CH₂)₃— and B is —(CH₂)₇—.

In one embodiment, the compound is a polyaminoguanidine of the formula (I):

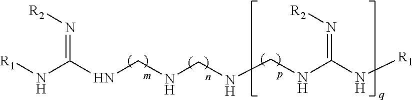

or a salt, solvate, or hydrate thereof, wherein n is an integer from 1 to 12, m and p are independently an integer from 1 to 5, q is 0 or 1, each R₁ is independently selected from the group consisting of C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, C₇-C₂₄ substituted or unsubstituted heteroaralkyl or C₁-C₈ alkyl-CH═C═CH₂ or C₁-C₈ alkyl-C≡CH or C₁-C₈ alkyl-cyclopropane or C(═O)C₁-C₈alkyl substituted with at least one halo group or cyclopropyl-NR₂ where each R is independently H, alkyl, or aralkyl, with the proviso that at least one R₁ is C₁-C₈ alkyl-CH═C═CH₂, C₁-C₈ alkyl-C≡CH, C₁-C₈ alkyl-cyclopropane, or C(═O)C₁-C₈ alkyl substituted with at least one halo group or cyclopropyl-NR₂ where each R is independently H, alkyl, or aralkyl; and each R₂ is independently selected from hydrogen or a C₁-C₈ substituted or unsubstituted alkyl.

In another embodiment, at least one R₁ is C₁-C₈ alkyl-CH═C═CH₂. In one embodiment, the at least one R₁ that is C₁-C₈ alkyl-CH═C═CH₂ is C₁-C₈ n-alkyl-CH═C═CH₂. In another embodiment, the at least one R₁ that is C₁-C₈ alkyl-CH═C═CH₂ is —CH₂—CH═C═CH₂.

In another embodiment, at least one R₁ is C₁-C₈ alkyl-C≡CH. In one embodiment, the at least one R₁ that is C₁-C₈ alkyl-C≡CH is C₁-C₈ n-alkyl-C≡CH. In another embodiment, the at least one R₁ that is C₁-C₈ alkyl-C≡CH is propargyl (CH₂—C≡CH).

In another embodiment, at least one R₁ is C₁-C₈ alkyl-cyclopropane. In one embodiment, the at least one R₁ that is C₁-C₈ alkyl-cyclopropane is C₁-C₈ n-alkyl-cyclopropane. In another variation, the at least one R₁ that is C₁-C₈ alkyl-cyclopropane is cyclopropylmethyl

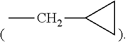

In another embodiment, at least one of R₁ is C(═O)C₁-C₈alkyl substituted with at least one halo group. In one embodiment, the at least one of R₁ that is C(═O)C₁-C₈alkyl substituted with at least one halo group is C(═O)C₁-C₈ n-alkyl substituted with at least one halo group selected from chloro or fluoro. In one embodiment, the at least one of R₁ that is C(═O)C₁-C₈alkyl substituted with at least one halo group is C(═O)C₁-C₄ n-alkyl substituted with at least one chloro group. In another embodiment, the at least one of R₁ that is C(═O)C₁-C₈alkyl substituted with at least one halo group is C(═O)(CH₂)ₙCH₂Cl where n is 1-7. In another embodiment, the at least one of R₁ that is C(═O)C₁-C₈alkyl substituted with at least one halo group is chloromethylcarbonyl (C(═O)CH₂Cl).

In another embodiment, at least one of R₁ is cyclopropyl-NR₂ where each R is independently H, alkyl, or aralkyl. In one embodiment, the at least one R₁ that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety of the aralkyl group is a C₁-C₈ alkyl, which may be a C₁-C₈ n-alkyl. In another embodiment, the at least one R₁ that is cyclopropyl-NR₂ where each R is independently H, alkyl, or aralkyl is 1-N-methylaminecycloprop-2-yl

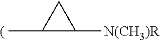

where R is C₁-C₈alkyl or aralkyl where the alkyl moiety of the aralkyl group is a C₁-C₈ alkyl). In another embodiment, the at least one R₁ that is cyclopropyl-NR₂ where each R is independently H, alkyl, or aralkyl is 1-N,N-dimethylaminocycloprop-2-yl.

In one embodiment, the compound is of the formula (I) wherein one R₁ is a C₆-C₂₀ substituted or unsubstituted aryl, such as a single ring substituted or unsubstituted aryl, including without limitation, substituted or unsubstituted phenyl. In one embodiment, the compound is of the formula (I) and each R₁ is phenyl. In one embodiment, q is 1, m and p are 3, and n is 4. In another embodiment, q is 1, m and p are 3, and n is 7.

In one embodiment, the compound is of the formula (I) wherein one R₁ is a C₈-C₁₂ or a C₁-C₈ substituted or unsubstituted alkyl, such as a linear alkyl. R₁ may be a C₁-C₈ substituted or unsubstituted linear alkyl, such as methyl or ethyl. In one embodiment, R₁ is methyl. R₁ may comprise or be a C₄-C₁₅ cycloalkyl group, such as a cycloalkyl group containing a linear alkyl group, where the cycloalkyl group is connected to the molecule either via its alkyl or cycloalkyl moiety. For instance, R₁ may be cyclopropylmethyl or cyclohexylmethyl. In one embodiment, R₁ is a C₃-C₁₅ branched alkyl group such as isopropyl. When R₁ is a C₁-C₈ substituted alkyl, the substituted alkyl may be substituted with any substituent, including a primary, secondary, tertiary or quaternary amine. Accordingly, in one embodiment, R₁ is a C₁-C₈ alkyl group substituted with an amine such that R₁ may be e.g., alkyl-NH₂ or an alkyl-amine-alkyl moiety such as —(CH₂)_{y}NH(CH₂)zCH₃ where y and z are independently an integer from 1 to 8. In one embodiment, R₁ is —(CH₂)₃NH₂.

In one embodiment, the compound is of the formula (I) where one R₁ is a C₇-C₂₄ substituted or unsubstituted aralkyl, which in one embodiment is an aralkyl connected to the molecule via its alkyl moiety (e.g., benzyl). In one embodiment, each R₁ is an aralkyl moiety wherein the alkyl portion of the moiety is substituted with two aryl groups and the moiety is connected to the molecule via its alkyl group. For instance, in one embodiment at least one or both R₁ is a C₇-C₂₄ aralkyl wherein the alkyl portion is substituted with two phenyl groups, such as when R₁ is 2,2-diphenylethyl or 2,2-dibenzylethyl. In one embodiment, each R₁ of formula (I) is 2,2-diphenylethyl and n is 1, 2 or 5. In one embodiment, each R₁ of formula (I) is 2,2-diphenylethyl, n is 1, 2 or 5 and m and p are each 1.

In one embodiment, at least one R₁ is hydrogen. When at least one R₁ is hydrogen, the other R₁ may be any moiety listed above for R₁, including an aryl group such as benzyl.

Any of the compounds of formula (I) listed above include compounds where at least one or both of R₂ is hydrogen or a C₁-C₈ substituted or unsubstituted alkyl.

In one embodiment, each R₂ is an unsubstituted alkyl such as methyl. In another embodiment, each R₂ is hydrogen.

Any of the compounds of formula (I) listed above may be compounds where q is 1 and m and p are the same. Accordingly, the polyaminoguanidines of formula (I) may be symmetric with reference to the polyaminoguanidine core (e.g., excluding R₁). Alternatively, the compounds of formula (I) may be asymmetric, e.g., when q is 0. In one embodiment, m and p are 1. In one embodiment, q is 0. In one embodiment, n is an integer from 1 to 5.

It is understood and clearly conveyed by this disclosure that each R₁, R₂, m, n, p and q disclosed in reference to formula (I) intends and includes all combinations thereof the same as if each and every combination of R₁, R₂, m, n, p and q were specifically and individually listed.

In one embodiment, the compound is a polyaminobiguanide or N-alkylated polyaminobiguanide. An N-alkylated polyaminobiguanide intends a polyaminobiguanide wherein at least one imine nitrogen of at least one biguanide is alkylated. In one embodiment, the compound is a polyaminobiguanide of the formula (II):

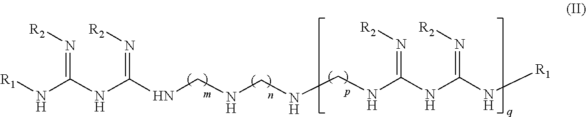

or a salt, solvate, or hydrate thereof, wherein n is an integer from 1 to 12, m and p are independently an integer from 1 to 5, q is 0 or 1, each R₁ is independently selected from the group consisting of C₁-C₈ substituted or unsubstituted alkyl, C₆-C₂₀ substituted or unsubstituted aryl, C₆-C₂₀ substituted or unsubstituted heteroaryl, C₇-C₂₄ substituted or unsubstituted aralkyl, C₇-C₂₄ substituted or unsubstituted heteroaralkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one R₁ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl.

In one embodiment, one of R₁ is a C₁-C₈ substituted or unsubstituted alkyl, such as those listed above in reference to formula (I). For instance, when R₁ is a C₁-C₈ substituted alkyl, the substituted alkyl may be substituted with any substituent, including a primary, secondary, tertiary or quaternary amine. Accordingly, in one embodiment, R₁ is a C₁-C₈ alkyl group substituted with an amine such that R₁ may be e.g., alkyl-NH₂ or an alkyl-amine-alkyl moiety such as —(CH₂)_{y}NH(CH₂)zCH₃ where y and z are independently an integer from 1 to 8. In one embodiment, R₁ is —(CH₂)₃NH₂. R₁ may also be a C₄-C₁₅ substituted or unsubstituted cycloalkyl or a C₃-C₁₅ substituted or unsubstituted branched alkyl, such as described for formula (I) above. In one embodiment, one of R₁ is a C₆-C₂₀ substituted or unsubstituted aryl, such as those listed above in reference to formula (I). In one embodiment, q is 1, m and p are 3, and n is 4. In another embodiment, q is l, m and p are 3, and n is 7.

In another embodiment, at least one R₁ is C₁-C₈ alkyl-CH═C═CH₂. In one embodiment, the at least one R₁ that is C₁-C₈ alkyl-CH═C═CH₂ is C₁-C₈ n-alkyl-CH═C═CH₂. In another embodiment, the at least one R₁ that is C₁-C₈ alkyl-CH═C═CH₂ is —CH₂—CH═C═CH₂.

In another embodiment, at least one R₁ is C₁-C₈ alkyl-C≡CH. In one embodiment, the at least one R₁ that is C₁-C₈ alkyl-C≡CH is C₁-C₈ n-alkyl-C≡CH. In another embodiment, the at least one R₁ that is C₁-C₈ alkyl-C≡CH is propargyl (CH₂—C≡CH).

In another embodiment, at least one R₁ is C₁-C₈ alkyl-cyclopropane. In one embodiment, the at least one R₁ that is C₁-C₈ alkyl-cyclopropane is C₁-C₈ n-alkyl-cyclopropane. In another variation, the at least one R₁ that is C₁-C₈ alkyl-cyclopropane is cyclopropylmethyl

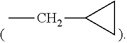

In another embodiment, at least one of R₁ is C(═O)C₁-C₈alkyl substituted with at least one halo group. In one embodiment, the at least one of R₁ that is C(═O)C₁-C₈alkyl substituted with at least one halo group is C(═O)C₁-C₈ n-alkyl substituted with at least one halo group selected from chloro or fluoro. In one embodiment, the at least one of R₁ that is C(═O)C₁-C₈alkyl substituted with at least one halo group is C(═O)C₁-C₄ n-alkyl substituted with at least one chloro group. In another embodiment, the at least one of R₁ that is C(═O)C₁-C₈alkyl substituted with at least one halo group is C(═O)(CH₂)ₙCH₂Cl where n is 1-7. In another embodiment, the at least one of R₁ that is C(═O)C₁-C₈alkyl substituted with at least one halo group is chloromethylcarbonyl (C(═O)CH₂Cl).

In another embodiment, at least one of R₁ is cyclopropyl-NR₂ where each R is independently H, alkyl, or aralkyl. In one embodiment, the at least one R₁ that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety

In one embodiment, the compound is of the formula (II) where at least one or both R₁ is a C₇-C₂₄ substituted or unsubstituted aralkyl, which in one embodiment is an aralkyl connected to the molecule via its alkyl moiety. In one embodiment, each R₁ is an aralkyl moiety wherein the alkyl portion of the moiety is substituted with one or two aryl groups and the moiety is connected to the molecule via its alkyl moiety. For instance, in one embodiment at least one or both R₁ is an aralkyl wherein the alkyl portion is substituted with two phenyl or benzyl groups, such as when R₁ is 2,2-diphenylethyl or 2,2-dibenzylethyl. In one embodiment, each R₁ of formula (II) is 2,2-diphenylethyl and n is 1, 2 or 5. In one embodiment, each R₁ of formula (II) is 2,2-diphenylethyl and n is 1, 2 or 5 and m and p are each 1.

Any of the compounds of formula (II) listed above include compounds where at least one or both of R₂ is hydrogen or a C₁-C₈ substituted or unsubstituted alkyl.

In one embodiment, each R₂ is an unsubstituted alkyl, such as methyl. In another embodiment, each R₂ is a hydrogen.

Any of the compounds of formula (II) listed above include compounds where q is 1 and m and p are the same. Accordingly, the polyaminobiguanides of formula (II) may be symmetric with reference to the polyaminobiguanide core (e.g., excluding R₁). Alternatively, the compounds of formula (II) may be asymmetric, e.g., when q is 0. In one embodiment, m and p are 1. In one embodiment, q is 0. In one embodiment, n is an integer from 1 to 5. In one embodiment, q, m and p are each 1 and n is 1, 2 or 5.

It is understood and clearly conveyed by this disclosure that each R₁, R₂, m, n, p and q disclosed in reference to formula (II) intends and includes all combinations thereof the same as if each and every combination of R₁, R₂, m, n, p and q were specifically and individually listed.

In one embodiment, the compound is a polyamine. In one embodiment, the polyamine is of the formula (III):

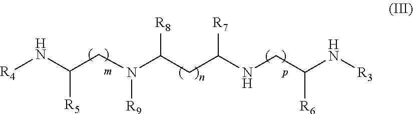

or a salt, solvate, or hydrate thereof, wherein n is an integer from 1 to 12; m and p are independently an integer from 1 to 5; R₃ and R₄ are independently selected from the group consisting of hydrogen, C₁-C₈ substituted or unsubstituted alkyl, C₆-C₂₀ substituted or unsubstituted aryl, C₇-C₂₄ substituted or unsubstituted aralkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₃ and R₄ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; each R₂ is independently hydrogen or a C₁-C₈ substituted or unsubstituted alkyl; R₅, R₉, R₆, R₇ and R₈ are independently selected from the group consisting of hydrogen and C₁-C₈ substituted or unsubstituted alkyl; and wherein either m and p are not the same integer or at least one of R₅, R₉, R₆, R₇ and R₈ is a C₁-C₈ substituted or unsubstituted alkyl.

In one embodiment, R₉ is a C₁-C₈ substituted or unsubstituted alkyl. When R₉ is a C₁-C₈ substituted alkyl, the substituted alkyl may be substituted with any substituent, including a primary, secondary, tertiary or quaternary amine. Accordingly, in one embodiment, R₉ is a C₁-C₈ alkyl group substituted with an amine such that R₉ may be e.g., alkyl-NH₂ or an alkyl-amine-alkyl moiety such as —(CH₂)_{y}NH(CH₂)zCH₃ where y and z are independently an integer from 1 to 8. In one embodiment, R₉ is —(CH₂)₃NHCH₂CH₃.

In one embodiment, one of R₃ and R₄ is hydrogen.

In one embodiment, one of R₃ and R₄ is a C₁-C₈ substituted or unsubstituted alkyl, including without limitation a substituted or unsubstituted n-alkyl (such as n-pentyl), substituted or unsubstituted branched (C₃-C₈) alkyl (such as 2-methylbutyl) or substituted or unsubstituted (C₃-C₈) cycloalkyl (such as cyclohexylmethyl). Larger chain alkyl (linear, branched and cyclic) are also considered, such as a C₉-C₁₅ substituted or unsubstituted alkyl. Where one of R₃ and R₄ is a C₁-C₈ substituted or unsubstituted n-alkyl, the moiety may be any n-alkyl, such as methyl or ethyl. When one of R₃ and R₄ is a substituted alkyl, whether linear, branched or cyclic, the alkyl may be substituted with one or more substituents such as those listed under “Substituted alkyl” and includes alkyl substituted with any halogen, such as a monohaloalkyl, dihaloalkyl, trihaloalkyl or multihaloalkyl, including a perhalooalkyl, for example, perfluoroalkyl and percholoralkyl, such as trifluoromethyl or pentachloroethyl.

In one embodiment, one of R₃ and R₄ is a C₆-C₂₀ substituted or unsubstituted aryl. In one embodiment, one of R₃ and R₄ is a C₆-C₂₀ substituted aryl, which aryl groups may be substituted with one or more substituents such as those listed under “Substituted aryl.” In one embodiment, one of R₃ and R₄ is a C₆-C₂₀ substituted aryl, which aryl groups may be substituted with one or more alkyoxy (such as —OCH₃), alkyl (including a branched alkyl such as tert-butyl), or halo groups (such as fluoro). In one embodiment, one of R₃ and R₄ is a halo-substituted aryl or a halo-substituted aralkyl, such as 2,4,5-trifluorophenyl or 2,4,5-trifluorobenzyl. In one embodiment, one of R₃ and R₄ is a di-alkyl-monoalkoxy-substituted aryl or aralkyl, such as 4,5-di-tert-butyl-2-methoxybenzyl or 4,5-di-tert-butyl-2-methoxyphenyl.

In one embodiment, one of R₃ and R₄ is a C₇-C₂₄ substituted or unsubstituted aralkyl or heteroaralkyl such as an aralkyl or heteroaralkyl connected to the molecule via its alkyl moiety. In one embodiment, one of R₃ and R₄ is a substituted aralkyl or heteroaralkyl connected to the molecule via its alkyl moiety. A substituted aralkyl may be substituted with one or more substituents such as those listed under “Substituted aralkyl” and a substituted heteroaralkyl may be substituted with one or more substituents such as those listed under “Substituted heteroaralkyl.” In one embodiment, one of R₃ and R₄ is a substituted heteroaralkyl having at least one nitrogen atom. In one embodiment, one of R₃ and R₄ is a single ring heteroaralkyl having at least one nitrogen atom. In one embodiment, one or both of R₃ and R₄ is 1-(2-N-methylpyrrolyl)-methyl.

In another embodiment, at least one of R₃ and R₄ is C₁-C₈ alkyl-CH═C═CH₂. In one embodiment, the at least one of R₃ and R₄ that is C₁-C₈ alkyl-CH═C═CH₂ is C₁-C₈ n-alkyl-CH═C═CH₂. In another embodiment, the at least one of R₃ and R₄ that is C₁-C₈ alkyl-CH═C═CH₂ is —CH₂—CH═C═CH₂.

In another embodiment, at least one of R₃ and R₄ is C₁-C₈ alkyl-C≡CH. In one embodiment, the at least one of R₃ and R₄ that is C₁-C₈ alkyl-C≡CH is C₁-C₈ n-alkyl-C≡CH. In another embodiment, the at least one of R₃ and R₄ that is C₁-C₈ alkyl-C≡CH is propargyl (CH₂—C≡CH).

In another embodiment, at least one of R₃ and R₄ is C₁-C₈ alkyl-cyclopropane. In one embodiment, the at least one of R₃ and R₄ that is C₁-C₈ alkyl-cyclopropane is C₁-C₈ n-alkyl-cyclopropane. In another variation, the at least one of R₃ and R₄ that is C₁-C₈ alkyl-cyclopropane is cyclopropylmethyl

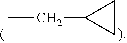

In another embodiment, at least one of R₃ and R₄ is C(═O)C₁-C₈alkyl substituted with at least one halo group. In one embodiment, the at least one of R₃ and R₄ that is C(═O)C₁-C₈alkyl substituted with at least one halo group is C(═O)C₁-C₈ n-alkyl substituted with at least one halo group selected from chloro or fluoro. In one embodiment, the at least one of R₃ and R₄ that is C(═O)C₁-C₈alkyl substituted with at least one halo group is C(═O)C₁-C₄ n-alkyl substituted with at least one chloro group. In another embodiment, the at least one of R₃ and R₄ that is C(═O)C₁-C₈alkyl substituted with at least one halo group is C(═O)(CH₂)ₙCH₂Cl where n is 1-7. In another embodiment, the at least one of R₃ and R₄ that is C(═O)C₁-C₈alkyl substituted with at least one halo group is chloromethylcarbonyl (C(═O)CH₂Cl).

In another embodiment, at least one of R₃ and R₄ is cyclopropyl-NR₂ where each R is independently H, alkyl, or aralkyl. In one embodiment, the at least one of R₃ and R₄ that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety of the aralkyl group is a C₁-C₈ alkyl, which may be a C₁-C₈ n-alkyl. In another embodiment, the at least one of R₃ and R₄ that is cyclopropyl-NR₂ where each R is independently H, alkyl, or aralkyl is 1-N-methylaminecycloprop-2-yl

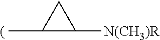

where R is C₁-C₈alkyl or aralkyl where the alkyl moiety of the aralkyl group is a C₁-C₈ alkyl). In another embodiment, the at least one of R₃ and R₄ that is cyclopropyl-NR₂ where each R is independently H, alkyl, or aralkyl is 1-N,N-dimethylaminocycloprop-2-yl.

In one embodiment, at least 1 or at least 2 or at least 3 of R₅, R₉, R₆, R₇ and R₈ is a C₁-C₈ substituted or unsubstituted alkyl. R₅, R₉, R₆, R₇ and R₈ may be a C₁-C₈ substituted or unsubstituted alkyl. In one embodiment at least 1 or at least 2 or at least 3 of R₅, R₉, R₆, R₇ is a C₁-C₈ unsubstituted n-alkyl, such as methyl or ethyl. In one embodiment, both R₆ and R₅ are methyl or ethyl. In one embodiment, at least one R₇ and R₈ is methyl or ethyl. In one embodiment, R₇ is methyl.

It is understood and clearly conveyed by this disclosure that each R₃, R₄, R₅, R₉, R₆, R₇, R₈, m, n, y, z and p disclosed in reference to formula (III) intends and includes all combinations thereof the same as if each and every combination of R₃, R₄, R₅, R₉, R₆, R₇, R₈, m, n, y, z and p were specifically and individually listed.

In one embodiment, the polyamine is of the formula (IV):

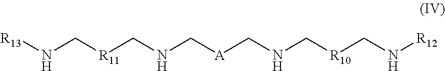

or a salt, solvate, or hydrate thereof, wherein A, R₁₀ and R₁₁ are independently (CH₂)ₙ or ethene-1,1-diyl; n is an integer from 1 to 5; R₁₂ and R₁₃ are independently selected from the group consisting of hydrogen, C₂-C₈ substituted or unsubstituted alkenyl, C₁-C₈ substituted or unsubstituted alkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₁₂ and R₁₃ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl. In one embodiment, at least one of A, R₁₀, R₁₁, R₁₂ and R₁₃ comprises an alkenyl moiety. In another embodiment, when any one or more of A, R₁₀, and R₁₁ is alkenyl, the alkene portion branches off the direct chain connecting the nitrogen atoms; that is, no more than one sp²-hybridized carbon occurs in the carbon nodes along the shortest path from one nitrogen flanking A, R₁₀, and/or R₁₁ to the other flanking nitrogen. For example, when A is ethene, the segment containing A is of the form —CH₂C(═CH₂)—CH₂— and the three nodes in the shortest carbon path between the nitrogens containing the A moiety has only one sp²-hybridized carbon. When A is propene, the segment containing A can be of the form —CH₂C(═CHCH₃)—CH₂— or —CH₂C(—CH═CH₂)—CH₂—.

In one embodiment, A is (CH₂)ₙ and n is 1. In one embodiment, A is ethene-1,1-diyl. In one embodiment, A is (CH₂)ₙ and one or both of R₁₂ and R₁₃ comprises an alkenyl moiety, such as propen-2-yl.

In one embodiment at least one or both of R₁₀ and R₁₁ is ethene-1,1-diyl. In one embodiment, both R₁₀ and R₁₁ are (CH₂)ₙ such as CH₂ (where n=1).

In one embodiment, at least one of R₁₂ and R₁₃ is hydrogen. In one embodiment, at least one of R₁₂ and R₁₃ is a C₂-C₈ substituted or unsubstituted alkenyl, such as propen-2-yl. In one embodiment, at least one of R₁₂ and R₁₃ is a C₁-C₈ substituted or unsubstituted alkyl, such as methyl or ethyl or any C₁-C₈ substituted or unsubstituted alkyl mentioned above in reference to any one of formulae (I), (II) or (III).

In another embodiment, at least one of R₁₂ and R₁₃ is C₁-C₈ alkyl-CH═C═CH₂. In one embodiment, the at least one of R₁₂ and R₁₃ that is C₁-C₈ alkyl-CH═C═CH₂ is C₁-C₈ n-alkyl-CH═C═CH₂. In another embodiment, the at least one of R₁₂ and R₁₃ that is C₁-C₈ alkyl-CH═C═CH₂ is —CH₂—CH═C═CH₂.

In another embodiment, at least one of R₁₂ and R₁₃ is C₁-C₉ alkyl-C≡CH. In one embodiment, the at least one of R₁₂ and R₁₃ that is C₁-C₈alkyl-C≡CH is C₁-C₈ n-alkyl-C≡CH. In another embodiment, the at least one of R₁₂ and R₁₃ that is C₁-C₈ alkyl-C≡CH is propargyl (CH₂—C≡CH).

In another embodiment, at least one of R₁₂ and R₁₃ is C₁-C₈ alkyl-cyclopropane. In one embodiment, the at least one of R₁₂ and R₁₃ that is C₁-C₈ alkyl-cyclopropane is C₁-C₈ n-alkyl-cyclopropane. In another variation, the at least one of R₁₂ and R₁₃ that is C₁-C₈ alkyl-cyclopropane is cyclopropylmethyl

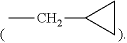

In another embodiment, at least one of R₁₂ and R₁₃ is C(═O)C₁-C₈alkyl substituted with at least one halo group. In one embodiment, the at least one of R₁₂ and R₁₃ that is C(═O)C₁-C₈alkyl substituted with at least one halo group is C(═O)C₁-C₈ n-alkyl substituted with at least one halo group selected from chloro or fluoro. In one embodiment, the at least one of R₁₂ and R₁₃ that is C(═O)C₁-C₈alkyl substituted with at least one halo group is C(═O)C₁-C₄ n-alkyl substituted with at least one chloro group. In another embodiment, the at least one of R₁₂ and R₁₃ that is C(═O)C₁-C₈alkyl substituted with at least one halo group is C(═O)(CH₂)ₙCH₂Cl where n is 1-7. In another embodiment, the at least one of R₁₂ and R₁₃ that is C(═O)C₁-C₈alkyl substituted with at least one halo group is chloromethylcarbonyl (C(═O)CH₂Cl).

In another embodiment, at least one of R₁₂ and R₁₃ is cyclopropyl-NR₂ where each R is independently H, alkyl, or aralkyl. In one embodiment, the at least one of R₁₂ and R₁₃ that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety of the aralkyl group is a C₁-C₈ alkyl, which may be a C₁-C₈ n-alkyl. In another embodiment, the at least one of R₁₂ and R₁₃ that is cyclopropyl-NR₂ where each R is independently H, alkyl, or aralkyl is 1-N-methylaminecycloprop-2-yl

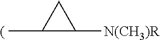

where R is C₁-C₈alkyl or aralkyl where the alkyl moiety of the aralkyl group is a C₁-C₈ alkyl). In another embodiment, the at least one of R₁₂ and R₁₃ that is cyclopropyl-NR₂ where each R is independently H, alkyl, or aralkyl is 1-N,N-dimethylaminocycloprop-2-yl.

It is understood and clearly conveyed by this disclosure that each A, n, R₁₀, R₁₁, R₁₂ and R₁₃ disclosed in reference to formula (IV) intends and includes all combinations thereof the same as if each and every combination of A, n, R₁₀, R₁₁, R₁₂ and R₁₃ were specifically and individually listed.

In one embodiment, the polyamine is of the formula (V):

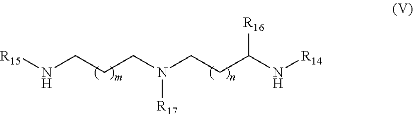

or a salt, solvate, or hydrate thereof, wherein n is an integer from 1 to 8; m is an integer from 1 to 8; R₁₅ and R₁₄ are independently selected from the group consisting of hydrogen, C₁-C₈ substituted or unsubstituted n-alkyl or (C₃-C₈) branched alkyl, C₆-C₂₀ substituted or unsubstituted aryl or heteroaryl, C₇-C₂₄ substituted or unsubstituted aralkyl or heteroaralkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₁₅ and R₁₄ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl; R₁₆ and R₁₇ are independently hydrogen or a C₁-C₈ substituted or unsubstituted alkyl; and wherein the compound contains no more than three secondary amino groups except when R₁₇ is a C₁-C₈ substituted or unsubstituted alkyl and wherein the compound is free from a methylphosphonate or hydroxy moiety.

In one embodiment, at least one of R₁₅ and R₁₄ is hydrogen. In one embodiment, at least one of R₁₅ and R₁₄ is a C₁-C₈ substituted or unsubstituted n-alkyl or (C₃-C₈) branched alkyl, such as methyl, ethyl, 3-methyl-butyl, 2-ethyl-butyl, 5-NH₂-pent-1-yl, prop-1-yl-methyl(phenyl)phosphinate and the like or any C₁-C₈ substituted or unsubstituted n-alkyl or (C₃-C₈) branched alkyl listed above in reference to formulae (I)-(IV). In one embodiment, at least one of R₁₅ and R₁₄ is a C₁-C₈ substituted or unsubstituted n-alkyl, such as an n-alkyl substituted with a methyl(phenyl)phosphinate moiety or a NH₂-substituted n-alkyl. In one embodiment, one of R₁₅ and R₁₄ is C₁-C₈ substituted or unsubstituted n-alkyl or (C₃-C₈) branched alkyl moieties, such as when one of R₁₅ and R₁₄ is 3-methyl-butyl or when one of R₁₅ and R₁₄ is 2-ethyl-butyl.

In one embodiment, at least one of R₁₅ and R₁₄ is a C₇-C₂₄ substituted or unsubstituted aralkyl or heteroaralkyl. In one embodiment, at least one of R₁₅ and R₁₄ is a C₇-C₂₄ substituted or unsubstituted aralkyl or heteroaralkyl having two rings, such as 2-phenylbenzyl, 4-phenylbenzyl, 2-benzylbenzyl, 3-benzylbenzyl, 3,3,-diphenylpropryl, 3-(benzoimidazolyl)-propyl and the like. In one embodiment, at least one of R₁₅ and R₁₄ is a C₇-C₂₄ substituted or unsubstituted aralkyl or heteroaralkyl having one ring, such as 4-isopropylbenzyl, 4-fluorobenzyl, 4-tert-butylbenzyl, 3-imidazolyl-propyl, 2-phenylethyl and the like. In one embodiment, one of R₁₅ and R₁₄ is a C₇-C₂₄ substituted or unsubstituted aralkyl or heteroaralkyl, such as any of the specific substituted or unsubstituted aralkyl or heteroaralkyl moieties listed for any other formula.

In another embodiment, at least one of R₁₅ and R₁₄ is C₁-C₈ alkyl-CH═C═CH₂. In one embodiment, the at least one of R₁₅ and R₁₄ that is C₁-C₈ alkyl-CH═C═CH₂ is C₁-C₈ n-alkyl-CH═C═CH₂. In another embodiment, the at least one of R₁₅ and R₁₄ that is C₁-C₈ alkyl-CH═C═CH₂ is —CH₂—CH═C═CH₂.

In another embodiment, at least one of R₁₅ and R₁₄ is C₁-C₈ alkyl-C≡CH. In one embodiment, the at least one of R₁₅ and R₁₄ that is C₁-C₈ alkyl-C≡CH is C₁-C₈ n-alkyl-C≡CH. In another embodiment, the at least one of R₁₅ and R₁₄ that is C₁-C₈ alkyl-C≡CH is propargyl (CH₂—C≡CH).

In another embodiment, at least one of R₁₅ and R₁₄ is C₁-C₈ alkyl-cyclopropane. In one embodiment, the at least one of R₁₅ and R₁₄ that is C₁-C₈ alkyl-cyclopropane is C₁-C₈ n-alkyl-cyclopropane. In another variation, the at least one of R₁₅ and R₁₄ that is C₁-C₈ alkyl-cyclopropane is cyclopropylmethyl

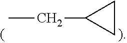

In another embodiment, at least one of R₁₅ and R₁₄ is C(═O)C₁-C₈alkyl substituted with at least one halo group. In one embodiment, the at least one of R₁₅ and R₁₄ that is C(═O)C₁-C₈alkyl substituted with at least one halo group is C(═O)C₁-C₈ n-alkyl substituted with at least one halo group selected from chloro or fluoro. In one embodiment, the at least one of R₁₅ and R₁₄ that is C(═O)C₁-C₈alkyl substituted with at least one halo group is C(═O)C₁-C₄ n-alkyl substituted with at least one chloro group. In another embodiment, the at least one of R₁₅ and R₁₄ that is C(═O)C₁-C₈alkyl substituted with at least one halo group is C(═O)(CH₂)ₙCH₂Cl where n is 1-7. In another embodiment, the at least one of R₁₅ and R₁₄ that is C(═O)C₁-C₈alkyl substituted with at least one halo group is chloromethylcarbonyl (C(═O)CH₂Cl).

In another embodiment, at least one of R₁₅ and R₁₄ is cyclopropyl-NR₂ where each R is independently H, alkyl, or aralkyl. In one embodiment, the at least one of R₁₅ and R₁₄ that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety of the aralkyl group is a C₁-C₈ alkyl, which may be a C₁-C₈ n-alkyl. In another embodiment, the at least one R₁ that is cyclopropyl-NR₂ where each R is independently H, alkyl, or aralkyl is 1-N-methylaminecycloprop-2-yl

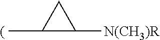

where R is C₁-C₈alkyl or aralkyl where the alkyl moiety of the aralkyl group is a C₁-C₈ alkyl). In another embodiment, the at least one of R₁₅ and R₁₄ that is cyclopropyl-NR₂ where each R is independently H, alkyl, or aralkyl is 1-N,N-dimethylaminocycloprop-2-yl.

For any compound of formula (V), m and n may be the same or different. In one embodiment, m does not equal n, such as when m is 1 and n is 2. For instance, in one embodiment, m is 1, and n is 2. However, it is understood that all possible combinations of m, n, R₁₅ and R₁₄ are intended.

In one embodiment, at least one or both of R₁₆ and R₁₇ is hydrogen. In one embodiment, at least one or both of R₁₆ and R₁₇ is a C₁-C₈ substituted or unsubstituted alkyl, such as a methyl, ethyl and a C₁-C₈ alkyl substituted with e.g., an —NH—C₁-C₈ alkyl such as when at least one or both of R₁₆ and R₁₇ is —(CH₂)₃NHCH₂CH₃.

It is understood and clearly conveyed by this disclosure that each R₁₄, R₁₅, R₁₆, R₁₇, m, and n disclosed in reference to formula (V) intends and includes all combinations thereof the same as if each and every combination of R₁₄, R₁₅, R₁₆, R₁₇, m, and n were specifically and individually listed.

In one embodiment, the polyamine is of the formula (VI):

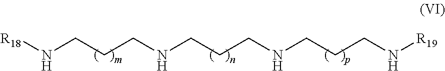

or a salt, solvate, or hydrate thereof, wherein n is an integer from 1 to 12; m and p are independently an integer from 1 to 5; R₁₈ and R₁₉ are independently selected from the group consisting of hydrogen, C₁-C₈ unsubstituted alkyl (e.g., methyl, ethyl, tert-butyl, isopropyl, pentyl, cyclobutyl), C₁-C₈ n-alkyl substituted with a cycloalkyl group comprising at least two rings, C₇-C₂₄ substituted or unsubstituted aralkyl or heteroaralkyl comprising at least two rings, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₁₈ and R₁₉ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl. In one variation, n is 1 when R₁₈ and R₁₉ are identical.

In one embodiment, at least one of R₁₈ and R₁₉ is a C₁-C₈ n-alkyl substituted with a cycloalkyl group comprising at least two rings. The cycloalkyl group comprising at least two rings may be a spiro, fused or bridged cycloalkyl group. Representative examples of a C₁-C₈ n-alkyl substituted with a cycloalkyl group comprising two rings include moieties such as 2-(6,6-dimethylbicyclo[3.1.1]heptyl)ethyl and 2-(decahydronaphthyl)ethyl. In one embodiment, at least one of R₁₈ and R₁₉ is 2-(6,6-dimethylbicyclo[3.1.1]heptyl)ethyl. In one embodiment, at least one of R₁₈ and R₁₉ are 2-(decahydronaphthyl)ethyl.

In one embodiment, at least one of R₁₈ and R₁₉ is a C₇-C₂₄ substituted or unsubstituted aralkyl or heteroaralkyl comprising at least two rings, which rings may be but are not required to be fused. A substituted aralkyl or heteroaralkyl with reference to formula (VI) intends and includes alkanoyl moieties substituted with an aryl or heteroaryl group, i.e., —C(═O)-aryl, —C(═O)-aralkyl, —C(═O)-heteroaryl, and —C(═O)-heteroaralkyl. In one embodiment, the alkyl portion of the aralkyl or heteroaralkyl moiety is connected to the molecule via its alkyl moiety. For instance at least one of R₁₈ and R₁₉ may be an aralkyl moiety such as 2-phenylbenzyl, 4-phenylbenzyl, 3,3,-diphenylpropyl, 2-(2-phenylethyl)benzyl, 2-methyl-3-phenylbenzyl, 2-napthylethyl, 4-(pyrenyl)butyl, 2-(3-methylnapthyl)ethyl, 2-(1,2-dihydroacenaphth-4-yl)ethyl and the like. In another embodiment, at least one of R₁₈ and R₁₉ may be a heteroaralkyl moiety such as 3-(benzoimidazolyl)propanoyl, 1-(benzoimidazolyl)methanoyl, 2-(benzoimidazolyl)ethanoyl, 2-(benzoimidazolyl)ethyl and the like.

In another embodiment, at least one of R₁₈ and R₁₉ is C₁-C₈ alkyl-CH═C═CH₂. In one embodiment, the at least one of R₁₈ and R₁₉ that is C₁-C₈ alkyl-CH═C═CH₂ is C₁-C₈ n-alkyl-CH═C═CH₂. In another embodiment, the at least one R₁ that is C₁-C₈ alkyl-CH═C═CH₂ is —CH₂—CH═C═CH₂.

In another embodiment, at least one of R₁₈ and R₁₉ is C₁-C₈alkyl-C≡CH. In one embodiment, the at least one of R₁₈ and R₁₉ that is C₁-C₈ alkyl-C≡CH is C₁-C₈ n-alkyl-C≡CH. In another embodiment, the at least one of R₁₈ and R₁₉ that is C₁-C₈ alkyl-C≡CH is propargyl (CH₂—C≡CH).

In another embodiment, at least one of R₁₈ and R₁₉ is C₁-C₈ alkyl-cyclopropane. In one embodiment, the at least one of R₁₈ and R₁₉ that is C₁-C₈ alkyl-cyclopropane is C₁-C₈ n-alkyl-cyclopropane. In another variation, the at least one of R₁₈ and R₁₉ that is C₁-C₈ alkyl-cyclopropane is cyclopropylmethyl

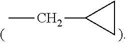

In another embodiment, at least one of R₁₈ and R₁₉ is C(═O)C₁-C₈alkyl substituted with at least one halo group. In one embodiment, the at least one of R₁₈ and R₁₉ that is C(═O)C₁-C₈alkyl substituted with at least one halo group is C(═O)C₁-C₈ n-alkyl substituted with at least one halo group selected from chloro or fluoro. In one embodiment, the at least one of R₁₈ and R₁₉ that is C(═O)C₁-C₈alkyl substituted with at least one halo group is C(═O)C₁-C₄ n-alkyl substituted with at least one chloro group. In another embodiment, the at least one of R₁₈ and R₁₉ that is C(═O)C₁-C₈alkyl substituted with at least one halo group is C(═O)(CH₂)ₙCH₂Cl where n is 1-7. In another embodiment, the at least one of R₁₈ and R₁₉ that is C(═O)C₁-C₈alkyl substituted with at least one halo group is chloromethylcarbonyl (C(═O)CH₂Cl).

In another embodiment, at least one of R₁₈ and R₁₉ is cyclopropyl-NR₂ where each R is independently H, alkyl, or aralkyl. In one embodiment, the at least one of R₁₈ and R₁₉ that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety of the aralkyl group is a C₁-C₈ alkyl, which may be a C₁-C₈ n-alkyl. In another embodiment, the at least one of R₁₈ and R₁₉ that is cyclopropyl-NR₂ where each R is independently H, alkyl, or aralkyl is 1-N-methylaminecycloprop-2-yl

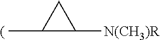

where R is C₁-C₈alkyl or aralkyl where the alkyl moiety of the aralkyl group is a C₁-C₈ alkyl). In another embodiment, the at least one of R₁₈ and R₁₉ that is cyclopropyl-NR₂ where each R is independently H, alkyl, or aralkyl is 1-N,N-dimethylaminocycloprop-2-yl.

In one embodiment, each of m, n and p is the same, such as when m, n and p are each 1.

It is understood and clearly conveyed by this disclosure that each R₁₈, R₁₉, m, n and p disclosed in reference to formula (VI) intends and includes all combinations thereof the same as if each and every combination of R₁₈, R₁₉, m, n and p were specifically and individually listed.

In one embodiment, the polyamine is of the formula (VII):

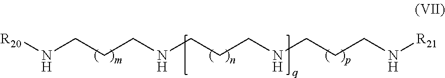

or a salt, solvate, or hydrate thereof, wherein n is an integer from 1 to 12; m and p are independently an integer from 1 to 5; q is 0 or 1; R₂₀ and R₂₁ are independently selected from the group consisting of hydrogen, C₁-C₈ substituted or unsubstituted alkyl, —C(═O)—C₁-C₈ substituted or unsubstituted alkyl, —C(═O)—C₁-C₈ substituted or unsubstituted alkenyl, —C(═O)—C₁-C₈ substituted or unsubstituted alkynyl, C₇-C₂₄ substituted or unsubstituted aralkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₂₀ and R₂₁ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl. In one embodiment, the compound also comprises at least one moiety selected from the group consisting of t-butyl, isopropyl, 2-ethylbutyl, 1-methylpropyl, 1-methylbutyl, 3-butenyl, isopent-2-enyl, 2-methylpropan-3-olyl, ethylthiyl, phenylthiyl, propynoyl, 1-methyl-1H-pyrrole-2-yl, trifluoromethyl, cyclopropanecarbaldehyde, halo-substituted phenyl, nitro-substituted phenyl, alkyl-substituted phenyl, 2,4,6-trimethylbenzyl, halo-S— substituted phenyl (such as para-(F₃S)-phenyl, azido and 2-methylbutyl.

In one embodiment, q is 1. In one embodiment, q is 1 and n is 1.

In one embodiment one of R₂₀ and R₂₁ is hydrogen. In one embodiment one of R₂₀ and R₂₁ is C₁-C₈ substituted or unsubstituted alkyl, such as any of the substituted or unsubstituted alkyl moieties mentioned above for formulas (I)-(VI). In one embodiment one of R₂₀ and R₂₁ is a C₇-C₂₄ substituted or unsubstituted aralkyl, such as any of the C₇-C₂₄ substituted or unsubstituted aralkyl mentioned above for formulas (I)-(VI).

In another embodiment, at least one of R₂₀ and R₂₁ is C₁-C₈ alkyl-CH═C═CH₂. In one embodiment, the at least one of R₂₀ and R₂₁ that is C₁-C₈ alkyl-CH═C═CH₂ is C₁-C₈ n-alkyl-CH═C═CH₂. In another embodiment, the at least one of R₂₀ and R₂₁ that is C₁-C₈ alkyl-CH═C═CH₂ is —CH₂—CH═C═CH₂.

In another embodiment, at least one of R₂₀ and R₂₁ is C₁-C₈ alkyl-C≡CH. In one embodiment, the at least one of R₂₀ and R₂₁ that is C₁-C₈ alkyl-C≡CH is C₁-C₈ n-alkyl-C≡CH. In another embodiment, the at least one of R₂₀ and R₂₁ that is C₁-C₈ alkyl-C≡CH is propargyl (CH₂—C≡CH).

In another embodiment, at least one of R₂₀ and R₂₁ is C₁-C₈ alkyl-cyclopropane. In one embodiment, the at least one of R₂₀ and R₂₁ that is C₁-C₈ alkyl-cyclopropane is C₁-C₈ n-alkyl-cyclopropane. In another variation, the at least one of R₂₀ and R₂₁ that is C₁-C₈ alkyl-cyclopropane is cyclopropylmethyl

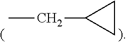

In another embodiment, at least one of R₂₀ and R₂₁ is C(═O)C₁-C₈alkyl substituted with at least one halo group. In one embodiment, the at least one of R₂₀ and R₂₁ that is C(═O)C₁-C₈alkyl substituted with at least one halo group is C(═O)C₁-C₈ n-alkyl substituted with at least one halo group selected from chloro or fluoro. In one embodiment, the at least one of R₂₀ and R₂₁ that is C(═O)C₁-C₈alkyl substituted with at least one halo group is C(═O)C₁-C₄ n-alkyl substituted with at least one chloro group. In another embodiment, the at least one of R₂₀ and R₂₁ that is C(═O)C₁-C₈alkyl substituted with at least one halo group is C(═O)(CH₂)ₙCH₂Cl where n is 1-7. In another embodiment, the at least one of R₂₀ and R₂₁ that is C(═O)C₁-C₈alkyl substituted with at least one halo group is chloromethylcarbonyl (C(═O)CH₂Cl).

In another embodiment, at least one of R₂₀ and R₂₁ is cyclopropyl-NR₂ where each R is independently H, alkyl, or aralkyl. In one embodiment, the at least one of R₂₀ and R₂₁ that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety of the aralkyl group is a C₁-C₈ alkyl, which may be a C₁-C₈ n-alkyl. In another embodiment, the at least one of R₂₀ and R₂₁ that is cyclopropyl-NR₂ where each R is independently H, alkyl, or aralkyl is 1-N-methylaminecycloprop-2-yl

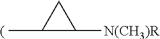

where R is C₁-C₈alkyl or aralkyl where the alkyl moiety of the aralkyl group is a C₁-C₈ alkyl). In another embodiment, the at least one of R₂₀ and R₂₁ that is cyclopropyl-NR₂ where each R is independently H, alkyl, or aralkyl is 1-N,N-dimethylaminocycloprop-2-yl.

It is understood and clearly conveyed by this disclosure that each R₂₀, R₂₁, m, n, q and p disclosed in reference to formula (VII) intends and includes all combinations thereof the same as if each and every combination of R₂₀, R₂₁, m, n, q and p were specifically and individually listed.

In one embodiment, the polyamine is of the formula (VIII):

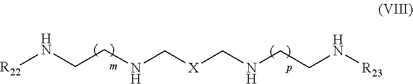

or a salt, solvate, or hydrate thereof, wherein m and p are independently an integer from 1 to 5; X is —(CH₂)n- or cyclohex-1,3-diyl; n is an integer from 1 to 5; R₂₂ and R₂₃ are independently selected from the group consisting of hydrogen, n-butyl, ethyl, cyclohexylmethyl, cyclopentylmethyl, cyclopropylmethyl, cycloheptylmethyl, cyclohexyleth-2-yl, benzyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₂₂ and R₂₃ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl. In one embodiment, n is 5, and at least one of R₂₂ and R₂₃ is hydrogen. In another embodiment, when X is cyclohex-1,3-diyl, R₂₂ and R₂₃ are not both benzyl or cyclopropylmethyl.

In one embodiment, X is —(CH₂)n (e.g., CH₂ where n is 1). In one embodiment, X is CH₂ and m and p are both 1. In one embodiment, X is cyclohex-1,3-diyl. In one embodiment, X is cyclohex-1,3-diyl and m and p are both 1. In other embodiments, m and p are not the same, e.g., when m is 3 and p is 4.

In another embodiment, at least one of R₂₂ and R₂₃ is C₁-C₈ alkyl-CH═C═CH₂. In one embodiment, the at least one of R₂₂ and R₂₃ that is C₁-C₈ alkyl-CH═C═CH₂ is C₁-C₈ n-alkyl-CH═C═CH₂. In another embodiment, the at least one of R₂₂ and R₂₃ that is C₁-C₈ alkyl-CH═C═CH₂ is —CH₂—CH═C═CH₂.

In another embodiment, at least one of R₂₂ and R₂₃ is C₁-C₈ alkyl-C≡CH. In one embodiment, the at least one of R₂₂ and R₂₃ that is C₁-C₈ alkyl-C≡CH is C₁-C₈ n-alkyl-C≡CH. In another embodiment, the at least one of R₂₂ and R₂₃ that is C₁-C₈ alkyl-C≡CH is propargyl (CH₂—C≡CH).

In another embodiment, at least one of R₂₂ and R₂₃ is C₁-C₈ alkyl-cyclopropane. In one embodiment, the at least one of R₂₂ and R₂₃ that is C₁-C₈ alkyl-cyclopropane is C₁-C₈ n-alkyl-cyclopropane. In another variation, the at least one of R₂₂ and R₂₃ that is C₁-C₈ alkyl-cyclopropane is cyclopropylmethyl

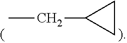

In another embodiment, at least one of R₂₂ and R₂₃ is C(═O)C₁-C₈alkyl substituted with at least one halo group. In one embodiment, the at least one of R₂₂ and R₂₃ that is C(═O)C₁-C₈alkyl substituted with at least one halo group is C(═O)C₁-C₈ n-alkyl substituted with at least one halo group selected from chloro or fluoro. In one embodiment, the at least one of R₂₂ and R₂₃ that is C(═O)C₁-C₈alkyl substituted with at least one halo group is C(═O)C₁-C₈ n-alkyl substituted with at least one chloro group. In another embodiment, the at least one of R₂₂ and R₂₃ that is C(═O)C₁-C₈alkyl substituted with at least one halo group is C(═O)(CH₂)ₙCH₂Cl where n is 1-7. In another embodiment, the at least one of R₂₂ and R₂₃ that is C(═O)C₁-C₈alkyl substituted with at least one halo group is chloromethylcarbonyl (C(═O)CH₂Cl).

In another embodiment, at least one of R₂₂ and R₂₃ is cyclopropyl-NR₂ where each R is independently H, alkyl, or aralkyl. In one embodiment, the at least one of R₂₂ and R₂₃ that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety of the aralkyl group is a C₁-C₈ alkyl, which may be a C₁-C₈ n-alkyl. In another embodiment, the at least one of R₂₂ and R₂₃ that is cyclopropyl-NR₂ where each R is independently H, alkyl, or aralkyl is 1-N-methylaminecycloprop-2-yl

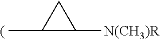

where R is C₁-C₈alkyl or aralkyl where the alkyl moiety of the aralkyl group is a C₁-C₈ alkyl). In another embodiment, the at least one of R₂₂ and R₂₃ that is cyclopropyl-NR₂ where each R is independently H, alkyl, or aralkyl is 1-N,N-dimethylaminocycloprop-2-yl.

It is understood and clearly conveyed by this disclosure that each R₂₂, R₂₃, m, n and p disclosed in reference to formula (VIII) intends and includes all combinations thereof the same as if each and every combination of R₂₂, R₂₃, m, n and p were specifically and individually listed.

In one embodiment, the polyamine is of the formula (IX):

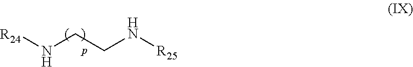

or a salt, solvate, or hydrate thereof, wherein p is an integer from 1 to 5; R₂₄ is an amino-substituted cycloalkyl (e.g., a cycloalkyl group substituted with a primary, secondary, tertiary or quaternary amine), a C₂-C₈ substituted or unsubstituted alkanoyl (which substituted alkanoyl may be substituted with one or more substituents such as those listed for “Substituted alkyl” including without limitation an alkanoyl substituted with a methyl and an alkylazide group), C₁-C₈ alkyl-CH═C═CH₂, C₁-C₈alkyl-C≡CH, C₁-C₈alkyl-cyclopropane, C(═O)C₁-C₈alkyl substituted with at least one halo group and cyclopropyl-NR₂ where each R is independently H, alkyl, or aralkyl; and R₂₅ is a C₁-C₈ substituted or unsubstituted alkyl, a C₇-C₂₄ substituted or unsubstituted aralkyl, such as those listed above for any of formulae (I)-(VIII), C₁-C₈ alkyl-CH═C═CH₂, C₁-C₈alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl with the proviso that at least one of R₂₄ and R₂₅ is C₁-C₈ alkyl-CH═C═CH₂ or C₁-C₈alkyl-C≡CH or C₁-C₈ alkyl-cyclopropane or C(═O)C₁-C₈alkyl substituted with at least one halo group or cyclopropyl-NR₂ where each R is independently H, alkyl, or aralkyl; and each R₂ is independently hydrogen or a C₁-C₈ substituted or unsubstituted alkyl.

In one embodiment, R₂₄ is an amino-substituted C₃-C₂₄ cycloalkyl, such as 5-NH₂-cycloheptyl, 3-NH₂-cyclopentyl and the like. In one embodiment, R₂₅ is a C₁-C₈ substituted or unsubstituted alkyl, which includes an n-alkyl group substituted with a cycloalkyl, such as in cyclopropylmethyl. In one embodiment, R₂₅ is cyclopropylmethyl or ethyl. In one embodiment, R₂₄ is 5-NH₂-cycloheptyl or 3-NH₂-cyclopentyl. In one embodiment, R₂₄ is a C₂-C₈ substituted or unsubstituted alkanoyl or R₂₄ is a C₇-C₂₄ substituted or unsubstituted aralkyl, such as 4-phenylbenzyl.

In another embodiment, at least one of R₂₄ and R₂₅ is C₁-C₈ alkyl-CH═C═CH₂. In one embodiment, the at least one of R₂₄ and R₂₅ that is C₁-C₈ alkyl-CH═C═CH₂ is C₁-C₈ n-alkyl-CH═C═CH₂. In another embodiment, the at least one of R₂₄ and R₂₅ that is C₁-C₈ alkyl-CH═C═CH₂ is —CH₂—CH═C═CH₂.

In another embodiment, at least one of R₂₄ and R₂₅ is C₁-C₈ alkyl-C≡CH. In one embodiment, the at least one of R₂₄ and R₂₅ that is C₁-C₈ alkyl-C≡CH is C₁-C₈ n-alkyl-C≡CH. In another embodiment, the at least one of R₂₄ and R₂₅ that is C₁-C₈ alkyl-C≡CH is propargyl (CH₂—C≡CH).

In another embodiment, at least one of R₂₄ and R₂₅ is C₁-C₈ alkyl-cyclopropane. In one embodiment, the at least one of R₂₄ and R₂₅ that is C₁-C₈ alkyl-cyclopropane is C₁-C₈ n-alkyl-cyclopropane. In another variation, the at least one of R₂₄ and R₂₅ that is C₁-C₈ alkyl-cyclopropane is cyclopropylmethyl

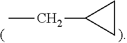

In another embodiment, at least one of R₂₄ and R₂₅ is C(═O)C₁-C₈alkyl substituted with at least one halo group. In one embodiment, the at least one of R₂₄ and R₂₅ that is C(═O)C₁-C₈alkyl substituted with at least one halo group is C(═O)C₁-C₈ n-alkyl substituted with at least one halo group selected from chloro or fluoro. In one embodiment, the at least one of R₂₄ and R₂₅ that is C(═O)C₁-C₈alkyl substituted with at least one halo group is C(═O)C₁-C₄ n-alkyl substituted with at least one chloro group. In another embodiment, the at least one of R₂₄ and R₂₅ that is C(═O)C₁-C₈alkyl substituted with at least one halo group is C(═O)(CH₂)ₙCH₂Cl where n is 1-7. In another embodiment, the at least one of R₂₄ and R₂₅ that is C(═O)C₁-C₈alkyl substituted with at least one halo group is chloromethylcarbonyl (C(═O)CH₂Cl).

In another embodiment, at least one of R₂₄ and R₂₅ is cyclopropyl-NR₂ where each R is independently H, alkyl, or aralkyl. In one embodiment, the at least one of R₂₄ and R₂₅ that is cyclopropyl-NR₂ where each R is independently H, alkyl, or aralkyl is cyclopropyl-NR₂ where each R is independently H, C₁-C₈ alkyl, which may be a C₁-C₈ n-alkyl, or aralkyl where the alkyl moiety of the aralkyl group is a C₁-C₈ alkyl, which may be a C₁-C₈ n-alkyl. In another embodiment, the at least one R₁ that is cyclopropyl-NR₂ where each R is independently H, alkyl, or aralkyl is 1-N-methylaminecycloprop-2-yl

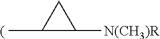

where R is C₁-C₈alkyl or aralkyl where the alkyl moiety of the aralkyl group is a C₁-C₈ alkyl). In another embodiment, the at least one of R₂₄ and R₂₅ that is cyclopropyl-NR₂ where each R is independently H, alkyl, or aralkyl is 1-N,N-dimethylaminocycloprop-2-yl.

It is understood and clearly conveyed by this disclosure that each R₂₄, R₂₅ and p disclosed in reference to formula (IX) intends and includes all combinations thereof the same as if each and every combination of R₂₄, R₂₅ and p were specifically and individually listed.

In one embodiment, the compound is of the formula (X):

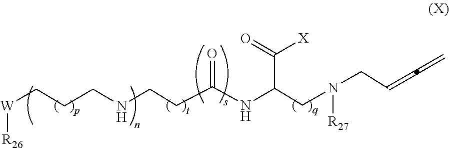

or a salt, solvate, or hydrate thereof, wherein R₂₆ is hydrogen, C₁-C₈alkyl or aralkyl where the alkyl moiety of the aryl group is a C₁-C₈alkyl, W is —NH—,

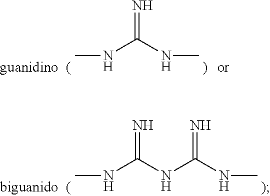

p and n are independently an integer from 1 to 5; t is an integer from 1 to 6; q is an integer from 1 to 10; s is 0 or 1; X is —O—C₁-C₈alkyl, OH or NHR₂₈, where R₂₈ is hydrogen, C₁-C₈alkyl or aralkyl where the alkyl moiety of the aryl group is a C₁-C₈alkyl; R₂₇ is hydrogen, C₁-C₈alkyl or aralkyl where the alkyl moiety of the aryl group is a C₁-C₈alkyl. In one embodiment, X is —OCH₃. In one embodiment, q is 3. In one embodiment, q is 4. In one embodiment, q is 5.

In one embodiment, the compound is of the formula (XI):

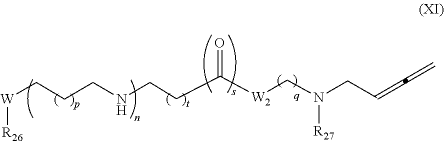

or a salt, solvate, or hydrate thereof, wherein R₂₆, W, p, n, t, s, q and R₂₇ are as defined for compound (X) and W₂ is —NH—,

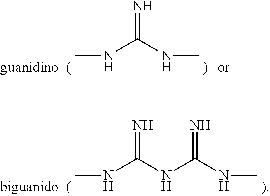

In one embodiment, the compound is of the formula (XII):

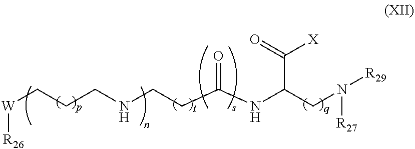

or a salt, solvate, or hydrate thereof, wherein R₂₆, W, p, n, t, s, X, q and R₂₇ are as defined for formula (X) and R₂₉ is C₁-C₈ alkyl-C≡CH, C₁-C₈cyclopropane or C(═O)C₁-C₈alkyl substituted with at least one halo group. In one embodiment, R₂₉ is propargyl, cyclopropylmethyl or chloromethylcarbonyl.

In one embodiment, the compound is of the formula (XIII):

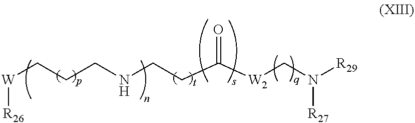

or a salt, solvate, or hydrate thereof, wherein R₂₆, W, W₂, p, n, t, s, q, R₂₇ and R₂₉ are as defined above.

In one embodiment, the compound is of the formula (XIV):

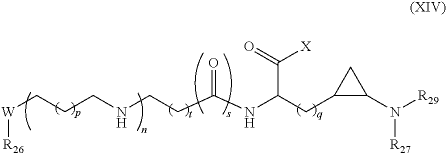

or a salt, solvate, or hydrate thereof, wherein R₂₆, W, p, n, t, s, q, R₂₇ and R₂₉ are as defined above.

In one embodiment, the compound is of the formula (XV):

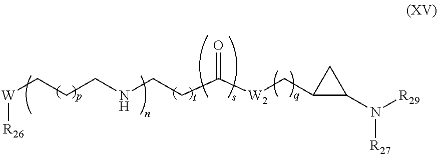

or a salt, solvate, or hydrate thereof, wherein R₂₆, W, W₂, p, n, t, s, q, R₂₃ and R₂₉ are as defined above.

In one embodiment, the compound is of the formula (XVI):

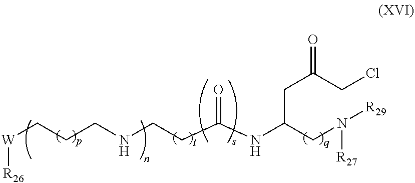

or a salt, solvate, or hydrate thereof, wherein R₂₆ is hydrogen, C₁-C₈alkyl or aralkyl where the alkyl moiety of the aryl group is a C₁-C₈alkyl, W is —NH—,

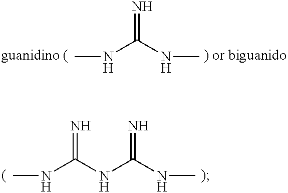

p and n are independently an integer from 1 to 5; t is an integer from 1 to 6; q is an integer from 1 to 10; s is 0 or 1; X is —O—C₁-C₈alkyl, OH or NHR₂₈, where R₂₈ is hydrogen, C₁-C₈alkyl or aralkyl where the alkyl moiety of the aryl group is a C₁-C₈alkyl; R₂₇ is hydrogen, C₁-C₈alkyl or aralkyl where the alkyl moiety of the aryl group is a C₁-C₈alkyl; and R₂₉ is C₁-C₈ alkyl-C≡CH, C₁-C₈cyclopropane or C(═O)C₁-C₈alkyl substituted with at least one halo group. In one embodiment, R₂₉ is propargyl, cyclopropylmethyl or chloromethylcarbonyl. In one embodiment, X is —OCH₃. In one embodiment, q is 3. In one embodiment, q is 4. In one embodiment, q is 5.

For all formulae listed herein, such as formulae (I)-(XVI), even if not explicitly stated, any substituent mentioned in one formula is intended to describe the same substituent in any other formula to the extent that the description conforms to the structural characterization of the formula described. For example, R₁, in formula I is intended to describe any other R₁ found in any other formula to the extent that the description conforms to the structural characterization of the formula described. Similarly, any description of, e.g., C₁-C₈ substituted or unsubstituted alkyl is intended to describe any other C₁-C₈ substituted or unsubstituted alkyl found in any other formula to the extent that the description conforms to the structural characterization of the formula described.

It is also recognized that any compounds listed as a particular salt thereof is not intended to limit the compound to such salt or form thereof. Similarly, where compounds are listed as a salt, the structure may or may not explicitly indicate positive or negative charges or the location thereof, and all possibilities thereof are intended. For instance, a compound listed as a 4HBr salt does not limit the compound to only the HBr salt and the compound may or may not show the + or − charges of the HBr salt, but rather all possibilities are intended.

Any of the polyamine compounds, such as compounds of the formula (I)-(IX) may be in a protected form, such as when any one or more amine (e.g., —NH—) is protected by a protecting group (Pg), such as in (—NPg-). Pg may be any protecting group, such as mesityl (e.g., NMes), Boc (e.g., —NBoc) or any other protecting group such as those described in, e.g. T. W. Green, P. G. M. Wuts, Protective Groups in Organic Synthesis, Wiley-Interscience, New York, 1999, which is incorporated herein by reference in its entirety.

In another embodiment, the invention embraces a method of treating cancer, by administering a therapeutically effective amount of one or more of the compounds of formula (M).

In another embodiment, the invention embraces a method of treating cancer, by administering a therapeutically effective amount of one or more of the compounds of formula (I).

In another embodiment, the invention embraces a method of treating cancer, by administering a therapeutically effective amount of one or more of the compounds of formula (II).

In another embodiment, the invention embraces a method of treating cancer, by administering a therapeutically effective amount of one or more of the compounds of formula (III).

In another embodiment, the invention embraces a method of treating cancer, by administering a therapeutically effective amount of one or more of the compounds of formula (IV).

In another embodiment, the invention embraces a method of treating cancer, by administering a therapeutically effective amount of one or more of the compounds of formula (V).

In another embodiment, the invention embraces a method of treating cancer, by administering a therapeutically effective amount of one or more of the compounds of formula (VI).

In another embodiment, the invention embraces a method of treating cancer, by administering a therapeutically effective amount of one or more of the compounds of formula (VII).

In another embodiment, the invention embraces a method of treating cancer, by administering a therapeutically effective amount of one or more of the compounds of formula (VIII).

In another embodiment, the invention embraces a method of treating cancer, by administering a therapeutically effective amount of one or more of the compounds of formula (IX).

In another embodiment, the invention embraces a method of treating cancer, by administering a therapeutically effective amount of one or more of the compounds of formula (X).

In another embodiment, the invention embraces a method of treating cancer, by administering a therapeutically effective amount of one or more of the compounds of formula (XI).

In another embodiment, the invention embraces a method of treating cancer, by administering a therapeutically effective amount of one or more of the compounds of formula (XII).

In another embodiment, the invention embraces a method of treating cancer, by administering a therapeutically effective amount of one or more of the compounds of formula (XIII).

In another embodiment, the invention embraces a method of treating cancer, by administering a therapeutically effective amount of one or more of the compounds of formula (XIV).

In another embodiment, the invention embraces a method of treating cancer, by administering a therapeutically effective amount of one or more of the compounds of formula (XV).

In another embodiment, the invention embraces a method of treating cancer, by administering a therapeutically effective amount of one or more of the compounds of formula (XVI).

In another embodiment, the invention embraces a method of inhibiting a histone demethylase enzyme, such as LSD1, by contacting the enzyme with an amount of one or more compounds, where the compound has an allene, propargyl, alkynyl, cyclopropyl, choromethyl ketone, and also at least one guanidine moiety or at least one biguanide moiety, in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (M) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (I) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (II) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (III) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (IV) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (V) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (VI) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (VII) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (VIII) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (IX) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (X) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (XI) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (XII) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (XIII) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (XIV) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (XV) in an amount sufficient to inhibit the enzyme. The enzyme can be inhibited by at least about 25%, at least about 50%, at least about 75%, at least about 90%, at least about 95%, or at least about 99%.

In another embodiment, the invention embraces a method of inhibiting a histone demethylase enzyme, such as LSD1, by contacting the enzyme with an amount of one or more of the compounds of formula (XVI) in an amount sufficient to inhibit the enzyme. The enzyme can be inhibited by at least about 25%, at least about 50%, at least about 75%, at least about 90%, at least about 95%, or at least about 99%.

### DETAILED DESCRIPTION OF THE INVENTION

The disclosure includes all salts of the compounds described herein. The invention also includes all non-salt compounds of any salt of a compound named herein, as well as other salts of any salt of a compound named herein. In one embodiment, the salts of the compounds comprise pharmaceutically acceptable salts. Pharmaceutically acceptable salts are those salts which retain the biological activity of the free compounds and which can be administered as drugs or pharmaceuticals to humans and/or animals. The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, can also be prepared.

The disclosure includes all solvates of the compounds described herein, such as hydrates (in any ratios, e.g. monohydrates, dihydrates, hemihydrates, sesquihydrates), methanolates, ethanolates, etc.

Any compound described herein may occur in a combined salt and solvate form, for example the hyclate (monohydrochloride hemiethanolate hemihydrate) form.

The disclosure includes all stereoisomers of the compounds described herein, including diastereomers and enantiomers in optically pure or substantially optically pure form, as well as mixtures of stereoisomers in any ratio, including, but not limited to, racemic mixtures. Unless stereochemistry is explicitly indicated in a chemical structure or chemical name, the chemical structure or chemical name is intended to embrace all possible stereoisomers of the compound depicted. For cyclopropyl groups, the structures are intended to embrace all stereoisomers of both cis- and trans-substituted cyclopropyl groups.

The disclosure includes all crystal and non-crystalline forms of the compounds described herein, including all polymorphs, polycrystalline, and amorphous forms and any mixtures thereof.

The term “alkyl” refers to saturated aliphatic and alicyclic groups including straight-chain, branched-chain, cyclic groups, and combinations thereof, having the number of carbon atoms specified, or if no number is specified, having up to 12 carbon atoms. “Straight-chain alkyl” or “linear alkyl” groups refers to alkyl groups that are neither cyclic nor branched, commonly designated as “n-alkyl” groups. C₁-C₈ n-alkyl consists of the following groups: —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, and —CH₂CH₂CH₂CH₂CH₂CH₂CH₂CH₂—. Other examples of alkyl groups include, but are not limited to, groups such as methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. Cycloalkyl groups can consist of one ring, including, but not limited to, groups such as cycloheptyl, or multiple bridged or fused rings, including, but not limited to, groups such as adamantyl or norbornyl groups. Cycloalkyl groups can also contain alkyl groups in addition to the cyclic portion, e.g., 2,6,6-trimethylbicyclo[3.1.1]heptane, 2-methyldecalin (2-methyldecahydronaphthalene), cyclopropylmethyl, cyclohexylmethyl, cycloheptylmethyl, and the like.

“Substituted alkyl” refers to alkyl groups substituted with one or more substituents including, but not limited to, groups such as halogen (including fluoro, chloro, bromo, and/or iodo-substituted alkyl such as a monohaloalkyl, dihaloalkyl, trihaloalkyl or multihaloalkyl, including a perhalooalkyl, for example, perfluoroalkyl, percholoralkyl, trifluoromethyl or pentachloroethyl), alkoxy, acyloxy, amino (including NH₂, NHalkyl and N(alkyl)₂), hydroxyl, mercapto, carboxy, benzyloxy, phenyl, benzyl, cyano, nitro, acyl, acylamino, amidino, alkyl amidino, thioamidino, aminoacyl, aryl, substituted aryl, aryloxy, azido, thioalkyl, —OS(O)₂-alkyl, thioalkoxy, carboxaldehyde, carboalkoxy and carboxamide, or a functionality that can be suitably blocked, if necessary for purposes of the invention, with a protecting group. Examples of substituted alkyl groups include, but are not limited to, CF₃, CF₂CF₃, and other perfluoro and perhalo groups; —CH₂—OH; —CH₂CH₂CH(NH₂)CH₃, etc. Alkyl groups can be substituted with other alkyl groups, e.g., C₃-C₂₄ cycloalkyl groups.

The term “alkenyl” refers to unsaturated aliphatic and alicyclic groups including straight-chain (linear), branched-chain, cyclic groups, and combinations thereof, having the number of carbon atoms specified, or if no number is specified, having up to 12 carbon atoms, which contain at least one double bond (—C═C—). Examples of alkenyl groups include, but are not limited to, —CH₂—CH═CH—CH₃; and —CH₂—CH₂-cyclohexenyl, where the ethyl group can be attached to the cyclohexenyl moiety at any available carbon valence. The term “alkynyl” refers to unsaturated aliphatic and alicyclic groups including straight-chain (linear), branched-chain, cyclic groups, and combinations thereof, having the number of carbon atoms specified, or if no number is specified, having up to 12 carbon atoms, which contain at least one triple bond (—C≡C—). “Hydrocarbon chain” or “hydrocarbyl” refers to any combination of straight-chain, branched-chain, or cyclic alkyl, alkenyl, or alkynyl groups, and any combination thereof. “Substituted alkenyl,” “substituted alkynyl,” and “substituted hydrocarbon chain” or “substituted hydrocarbyl” refer to the respective group substituted with one or more substituents, including, but not limited to, groups such as halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or any group listed above for “Substituted alkyl,” or a functionality that can be suitably blocked, if necessary for purposes of the invention, with a protecting group.

“Aryl” or “Ar” refers to an aromatic carbocyclic group having a single ring (including, but not limited to, groups such as phenyl), two or more rings connected to each other (including, but not limited to, groups such as biphenyl and p-diphenylbenzene) or two or more condensed rings (including, but not limited to, groups such as naphthyl, anthryl, or pyrenyl), and includes both unsubstituted and substituted aryl groups. Aryls, unless otherwise specified, contain from 6 to 20 carbon atoms in the ring portion. A preferred range for aryls contains 6 to 12 carbon atoms in the ring portion. “Substituted aryls” refers to aryls substituted with one or more substituents, including, but not limited to, groups such as substituted or unsubstituted alkyl, substituted or unsubstituted alkenyl, substituted or unsubstituted alkynyl, substituted or unsubstituted hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or any group listed above for “Substituted alkyl,” or a functionality that can be suitably blocked, if necessary for purposes of the invention, with a protecting group. “Aralkyl” designates an alkyl-substituted aryl group, where any aryl can be attached to the alkyl; the alkyl portion can comprise one, two, or three straight chains of 1 to 6 carbon atoms each or one, two, or three branched chains of 3 to 6 carbon atoms each or any combination thereof. Aralkyl groups can consist of two aryl groups connected by an alkyl group, such as diphenylmethane or 2-methyl-1-(phenethyl)benzene. When an aralkyl group is indicated as a substituent, the aralkyl group can be connected to the remainder of the molecule at any available valence on either its alkyl moiety or aryl moiety; e.g., the tolyl aralkyl group can be connected to the remainder of the molecule by replacing any of the five hydrogens on the aromatic ring moiety with the remainder of the molecule, or by replacing one of the alpha-hydrogens on the methyl moiety with the remainder of the molecule. Preferably, the aralkyl group is connected to the remainder of the molecule via the alkyl moiety.

A preferred aryl group is phenyl, which can be substituted or unsubstituted. Substituents for substituted phenyl groups include lower alkyl (—C₁-C₄ alkyl), or a halogen (chlorine (Cl), bromine (Br), iodine (I), or fluorine (F); hydroxy (—OH), or lower alkoxy (—C₁-C₄ alkoxy), such as methoxy, ethoxy, propyloxy(propoxy) (either n-propoxy or i-propoxy), and butoxy (either n-butoxy, i-butoxy, sec-butoxy, or tert-butoxy); a preferred alkoxy substituent is methoxy. Substituted phenyl groups preferably have one or two substituents; more preferably, one substituent. For aralkyl groups, a preferred group for the aryl portion is phenyl, which can be unsubstituted or substituted as described immediately above.

“Heteroalkyl,” “heteroalkenyl,” and “heteroalkynyl” refer to alkyl, alkenyl, and alkynyl groups, respectively, that contain the number of carbon atoms specified (or if no number is specified, having up to 12 carbon atoms) which contain one or more heteroatoms as part of the main, branched, or cyclic chains in the group. Heteroatoms include, but are not limited to, N, S, O, and P; N and O are preferred. Heteroalkyl, heteroalkenyl, and heteroalkynyl groups may be attached to the remainder of the molecule at any valence where a hydrogen can be removed, for example, at a heteroatom or at a carbon atom (if a valence is available at such an atom by removing a hydrogen). Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₂—, 1-ethyl-6-propylpiperidino, and morpholino. Examples of heteroalkenyl groups include, but are not limited to, groups such as —CH═CH—NH—CH(CH₃)—CH₂—. “Heteroaryl” or “HetAr” refers to an aromatic carbocyclic group having a single ring (including, but not limited to, examples such as pyridyl, imidazolyl, thiophene, or furyl) or two or more condensed rings (including, but not limited to, examples such as indolizinyl, indole, benzimidazole, benzotriazole, or benzothienyl) and having at least one hetero atom, including, but not limited to, heteroatoms such as N, O, P, or S, within the ring. Unless otherwise specified, heteroalkyl, heteroalkenyl, heteroalkynyl, and heteroaryl groups have between one and five heteroatoms and between one and twelve carbon atoms. “Substituted heteroalkyl,” “substituted heteroalkenyl,” “substituted heteroalkynyl,” and “substituted heteroaryl” groups refer to heteroalkyl, heteroalkenyl, heteroalkynyl, and heteroaryl groups substituted with one or more substituents, including, but not limited to, groups such as substituted or unsubstituted alkyl, substituted or unsubstituted alkenyl, substituted or unsubstituted alkynyl, substituted or unsubstituted benzyl, substituted or unsubstituted hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or any group listed above for “Substituted alkyl,” or a functionality that can be suitably blocked, if necessary for purposes of the invention, with a protecting group. Examples of such substituted heteroalkyl groups include, but are not limited to, piperazine, substituted at a nitrogen or carbon by a phenyl or benzyl group, and attached to the remainder of the molecule by any available valence on a carbon or nitrogen, —NH—SO₂-phenyl, —NH—(C═O)O-alkyl, —NH—(C═O)O-alkyl-aryl, and —NH—(C═O)-alkyl. If chemically possible, the heteroatom(s) and/or the carbon atoms of the group can be substituted. A “heteroaralkyl” group is a heteroaryl group substituted with at least one alkyl group. The heteroatom(s) can also be in oxidized form, if chemically possible.

The term “alkoxy” as used herein refers to an alkyl, alkenyl, alkynyl, or hydrocarbon chain linked to an oxygen atom and having the number of carbon atoms specified, or if no number is specified, having up to 12 carbon atoms. Examples of alkoxy groups include, but are not limited to, groups such as methoxy, ethoxy, propyloxy (propoxy) (either n-propoxy or i-propoxy), and butoxy (either n-butoxy, i-butoxy, sec-butoxy, or tert-butoxy).

The terms “halo” and “halogen” as used herein refer to the Group Vila elements (Group 17 elements in the 2005 IUPAC Periodic Table, IUPAC Nomenclature of Inorganic Chemistry) and include Cl, Br, F and I substituents.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1999) Protective Groups in Organic Synthesis, (Wiley-Interscience., New York). Amino protecting groups include, but are not limited to, mesitylenesulfonyl (Mts), benzyloxycarbonyl (CBz or Z), t-butyloxycarbonyl (Boc), t-butyldimethylsilyl(TBS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5 bromo 7-nitroindolinyl, and the like. Hydroxyl protecting groups include, but are not limited to, Fmoc, TBS, photolabile protecting groups (such as nitroveratryl oxymethyl ether (Nvom)), Mom (methoxy methyl ether), and Mem (methoxy ethoxy methyl ether), NPEOC (4-nitrophenethyloxycarbonyl) and NPEOM (4 nitrophenethyloxymethyloxycarbonyl).

### Synthetic Methods—Synthesis of Alkylpolyamines

Several synthetic methods are available for synthesis of polyamine analog compounds, including both symmetrically-substituted and asymmetrically-substituted polyamine analogs. Some of these methods are described in the following publications: Saab et al., J. Med. Chem. 36:2998 (1993); Bellevue et al., Bioorg. Med. Chem. Lett. 6:2765 (1996); Sirisoma et al., Tetrahedron Lett. 39:1489 (1998); Zou et al., Bioorg. Med. Chem. Lett. 11:1613 (2001), and Casero et al., J. Med. Chem. 44:1 (2001).

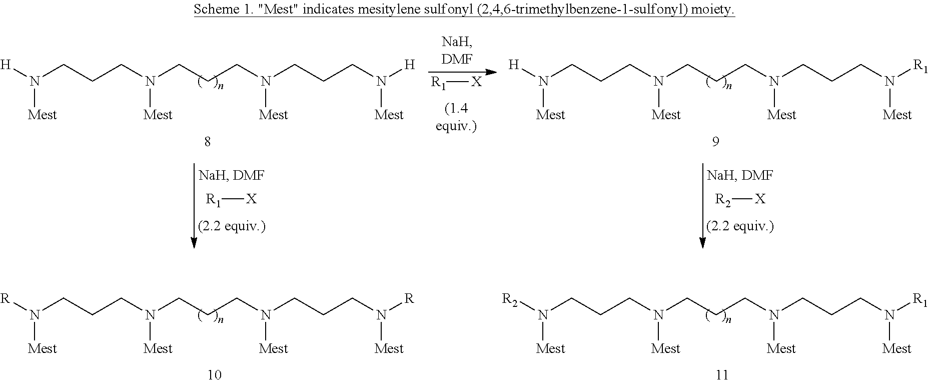

Scheme 1 illustrates a useful pathway to various polyamine analogs. The tetramesitylated intermediate 8 can be readily alkylated at both terminal nitrogens, since the hydrogens on these nitrogens are rendered acidic by the adjacent mesityl protecting group. Alkylation in the presence of 1.2 to 1.4 equivalents of alkyl halide or tosylate affords primarily the monosubstituted product 9, and disubstituted materials and unreacted starting material can then be separated and recycled (Bellevue et al., Bioorg. Med. Chem. Lett. 6:2765 (1996); Zou et al., Bioorg. Med. Chem. Lett. 11:1613 (2001)). The resulting monoalkylated derivative 9 can then be deprotected (30% HBr in AcOH), or realkylated with a different alkyl halide to provide the asymmetrically substituted intermediate 11. Deprotection of 11 then provides the desired asymmetrically substituted alkylpolyamine. Treatment of 8 with 2.2 equivalents of alkyl halide in the presence of NaH and DMF affords the bis-substituted intermediate 10, which upon deprotection yields the corresponding symmetrically substituted alkylpolyamine. Thus three distinct alkylpolyamines can be readily synthesized from a single intermediate, and the central carbon chain can be made in any desired length (n=0-8). Synthesis of the intermediate 8 is readily accomplished in large quantities using previously reported synthetic strategies (Bellevue et al., Bioorg. Med. Chem. Lett. 6:2765 (1996); Zou et al., Bioorg. Med. Chem. Lett. 11:1613 (2001)). A similar strategy can be used to access spermidine-like analogs of the form:

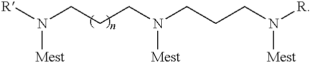

Other methods can be used for synthesis of the requisite polyamine backbone structures, which involve carbon nitrogen bond formation and selective nitrogen protection; some of these procedures are shown in Scheme 2.

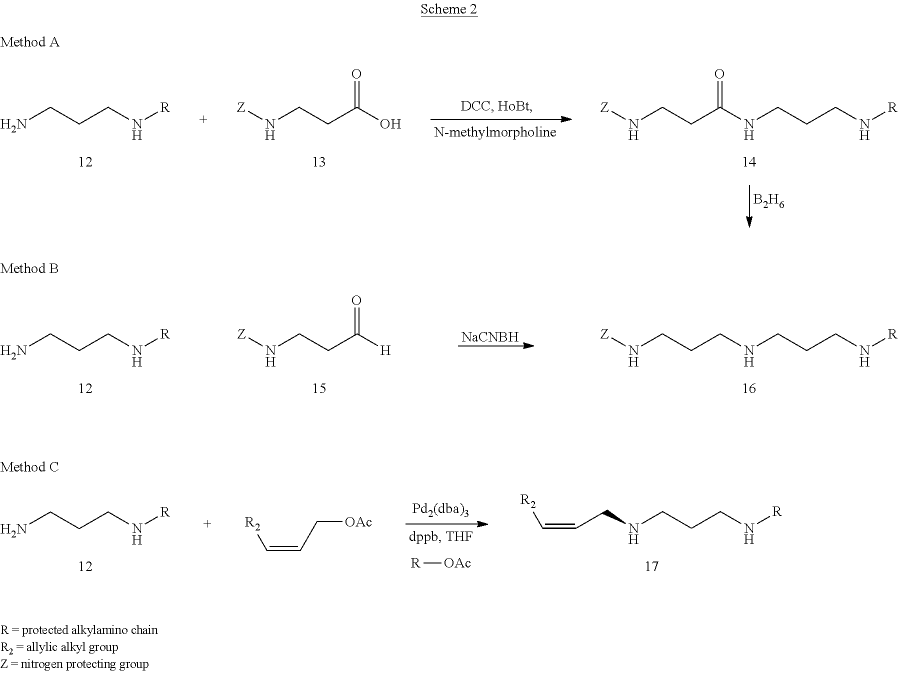

Aminopropyl (or other aminoalkyl) moieties can be added to selectively protected primary amines such as 12 by standard peptide coupling techniques (Method A, Woster et al., J. Med. Chem. 32:1300 (1989)). Thus treatment of 12 with the protected beta-aminopropionate 13 (DCC, HoBt, N-methylmorpholine) affords the corresponding amide 14, which is then reduced in the presence of diborane (Woster et al., 1989) to afford the desired secondary amine 16. Compound 16 may be synthesized directly by reductive amination (Method B), in which the appropriate aldehyde 15 is added to 12 in the presence of sodium cyanoborohydride. Alkyl substituents that contain an allylic acetate functionality can also be appended to 12 using a palladium catalyzed coupling reaction that proceeds with retention of configuration (Method C, Sirisoma et al., Tetrahedron Lett. 39:1489 (1998)). This method can also be used to introduce phthalimide or benzylamine to an allylic acetate site as a synthetic equivalent for nitrogen. These nitrogens can then be deprotected and functionalized.

### Synthetic Methods—Synthesis of Polyaminoguanidines

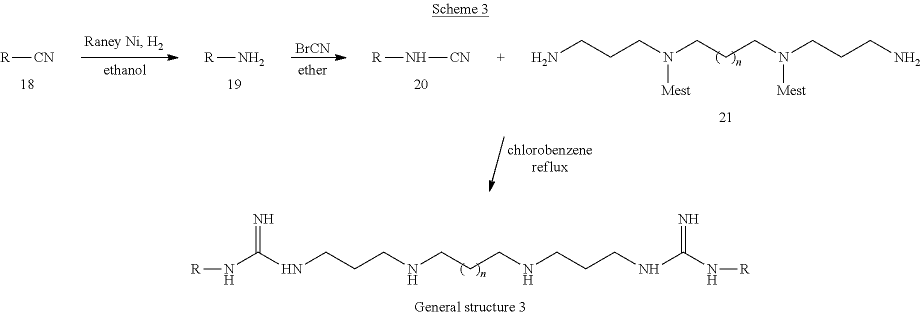

Synthesis of polyaminoguanidines can be carried out as outlined in Scheme 3. The requisite amine 19 (produced when necessary from the corresponding alkyl or aralkylcyanide) is reacted with cyanogen bromide (Goldin et al., U.S. Pat. No. 6,288,123 (2001)) to afford the corresponding aminocyanogen 20. When the desired amine is not commercially available, it can be prepared from the appropriate cyano compound by catalytic reduction (Bellevue et al., 1996, Zou et al., 2001). Intermediate 21 (Bellevue et al., 1996; Zou et al., 2001) is then coupled to 20 (chlorobenzene, reflux), followed by deprotection (30% Hbr in AcOH) to produce alkylpolyaminoguanidines of general structure 3. Using these methods, substituted polyaminoguanidine analogs (e.g., R═H, methyl, ethyl, cyclopropylmethylene, cycloheptylmethylene, phenyl, benzyl) can be synthesized. An analogous route (not shown) utilizing the N-Boc protection group was also employed.

### Synthetic Methods—Synthesis of Polyaminobiguanides

The synthesis of polyaminobiguanides is described in Bi et al., Bioorg. Med. Chem. Lett. 16:3229 (2006), and is also outlined in Scheme 4.

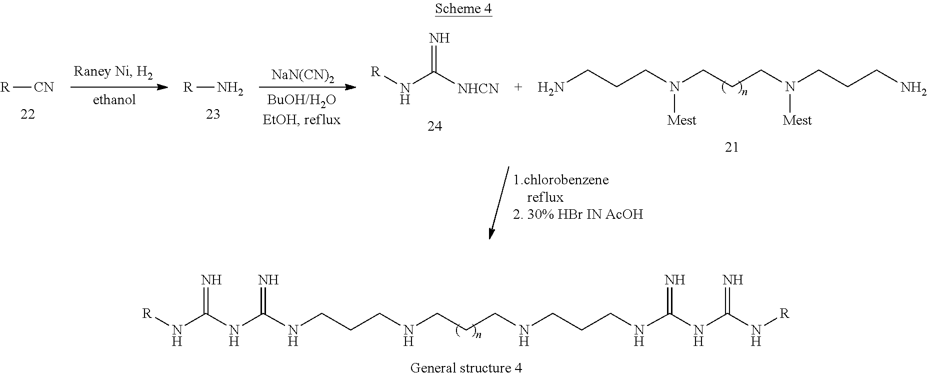

A similar strategy is employed for the synthesis of alkylpolyaminobiguanides of general structure 4, as outlined in Scheme 4. Amines 23 (produced when necessary from the corresponding alkyl or aralkylcyanide) are converted to the corresponding cyanoguanidines 24 (NaN(CN)₂, BuOH/H₂O) (Gerhard, R.; Heinz, B.; Herbert, F. J. Praktische Chem. (Leipzig), 1964, 26, 414-418), which were combined with 21 as previously described to afford the mesityl protected target molecules. Deprotection as described above then provided the substituted biguanides 4. An analogous route (not shown) utilizing the N-Boc protection group was also employed, as above.

### Synthetic Methods—Solid Phase Synthesis

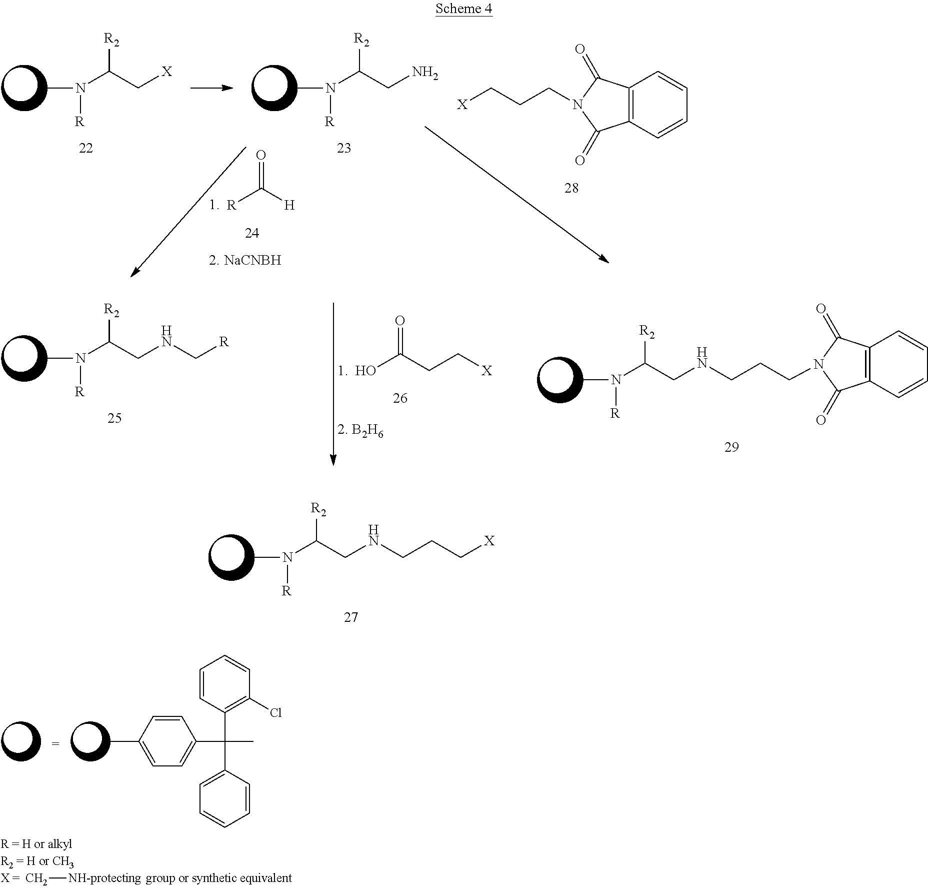

Solid phase synthetic techniques can be used for the rapid and efficient synthesis of both alkylpolyamines and their alpha-methyl homologs, as shown in Scheme 4 above. Compound 22 can be produced using a commercially available trityl chloride resin, as described in Wang et al., J. Am. Chem. Soc., 95(4):1328 (1973), where the attached amine is primary or secondary prior to attachment, an alpha-methyl is present or absent, and the X group is either a protected amine or a synthetic equivalent such as an azide or a phthalamide. This intermediate is then deprotected or converted to the corresponding primary amine 23. Three strategies can be used for chain elongation: 1. reductive amination with aldehydes 24 in the presence of sodium cyanoborohydride to produce 25; 2. addition of an appropriate carboxylate 26 under peptide coupling conditions (Woster et al., J. Med. Chem. 32:1300 (1989)), followed by diborane reduction of the resulting amide, yielding 27; 3. direct alkylation with a protected halide (Woster et al., J. Med. Chem. 32:1300 (1989)) such as 28, to afford intermediates 29. Repetition of these steps then allows the synthesis of a variety of alkylpolyamines and alpha-methyl-alkylpolyamines with substituents as desired.

### Synthesis of Compounds of Formula (X), Formula (XI), Formula (XII), Formula (XIII), Formula (XIV), Formula (XV), and Formula (XVI)

Synthesis of the compounds of formula (X) and formula (XI) can proceed via use of lysine-mimic synthons such as 107, 112 and 116 (Schemes 1-3). The synthesis of compound 107 is outlined in Scheme 1, starting from the α-N-Boc-lysine methyl ester 105 (R═C₁-C₈ alkyl, e.g., CH₃). The starting material 105 is conveniently prepared from α-N-Boc-lysine methyl ester (with an unprotected primary ε-amino group), commercially available from Bachem AG Biosciences. The ε-amino group can be alkylated, e.g., with a C₁-C₈ alkyl group or C₁-C₈ alkyl-C₆-C₁₀ aryl group, using an alkyl chloride, aralkyl chloride or other alkyl or aralkyl derivative reactive towards the amino group. Should an ester other than the methyl ester be desired, transesterification can be readily performed to replace the —OMe group with the desired ester, e.g., —O—C₁-C₈ alkyl. Alternatively, the ester can be easily converted to the —COOH group, which in turn can be reacted with an amine to form an amide group.

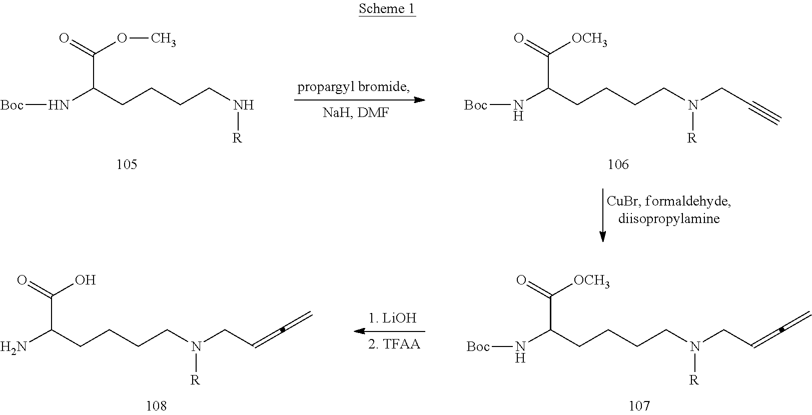

Compound 105 is reacted with propargyl bromide (NaH, DMF) (Bellevue, F. H. et al., Bioorg. Med. Chem. Lett., 6:2765-2770 (1996); Saab, N. H. et al., J. Med. Chem., 36:2998-3004 (1993)) to afford the alkylated amino acid 106. The acetylene moiety is then converted to the corresponding 2,3-butadiene moiety by treating it with cupric bromide and formaldehyde in the presence of diisopropylamine (Bey, P. et al., J. Med. Chem., 28:1-2 (1985)) to afford the desired synthon 107 (R═CH₃). Cleavage of the methyl ester (LiOH) (Bellevue, F. H. et al., Bioorg. Med. Chem. Lett., 6:2765-2770 (1996), Saab, N. H. et al., J. Med. Chem., 36:2998-3004 (1993), Varghese, S. et al., J. Med. Chem., 48:6350-6365 (2005)) produces the corresponding carboxylate substrate (as shown in 108) for coupling to an appropriate free amine (using DCC, HOBT) (Bellevue, F. H. et al., Bioorg. Med. Chem. Lett., 6:2765-2770 (1996), Saab, N. H. et al., J. Med. Chem., 36:2998-3004 (1993), Varghese, S. et al., J. Med. Chem., 48:6350-6365 (2005)), while removal of the N-Boc protecting group (with trifluoroacetic acid) (as shown in 108) affords a free amine useful for peptide coupling or reductive amination reactions (see, e.g., Casero, Jr. R. A. et al., J. Med. Chem., 44:1-26 (2001), Bellevue, F. H. et al., Bioorg. Med. Chem. Lett., 6:2765-2770 (1996), Saab, N. H. et al., J. Med. Chem., 36:2998-3004 (1993), and Varghese, S. et al., J. Med. Chem., 48:6350-6365 (2005)). A similar strategy, as outlined in Scheme 2, can be used to convert 1-N-(methyl)-5-N-(tertbutyloxycarbonyl)-1,5-diaminopentane 109 (R═CH₃) to the corresponding synthon 112 (R═CH₃). Treatment of 109 with propargyl bromide as described above results in intermediate 110, and subsequent conversion of the propargyl acetylene to the corresponding allene (Bey, P. et al., J. Med. Chem., 28:1-2 (1985)) then affords 111. (It should be noted that, when the propargyl group is desired instead of the allene group, this step can be omitted to obtain the propargyl-containing synthon). Removal of the N-Boc protecting group (e.g., with trifluoroacetic acid) then affords the desired free amine 112, which is also useful for peptide coupling or reductive amination reactions as indicated above. Finally, the O-methoxymethyl protected version of 1-hydroxy-5-[N-(methyl)]aminopentane 113 (Scheme 3) can be converted to the N-propargyl derivative 114, and then to the 2,3-butadienyl derivative 115, as described above (Bey, P. et al., J. Med. Chem., 28:1-2 (1985)), followed by acid deprotection of the alcohol and conversion of the corresponding hydroxyl group to a mesylate (Saab, N. H. et al., J. Med. Chem., 36:2998-3004 (1993)) to afford 116. Compound 116 can then be appended to a variety of aldehydes, amines, guanidines and guanides by nucleophilic substitution (see Casero, Jr. R. A. et al., J. Med. Chem., 44:1-26 (2001), Bellevue, F. H. et al., Bioorg. Med. Chem. Lett., 6:2765-2770 (1996), Saab, N. H. et al., J. Med. Chem., 36:2998-3004 (1993), and Varghese, S. et al., J. Med. Chem., 48:6350-6365 (2005)).

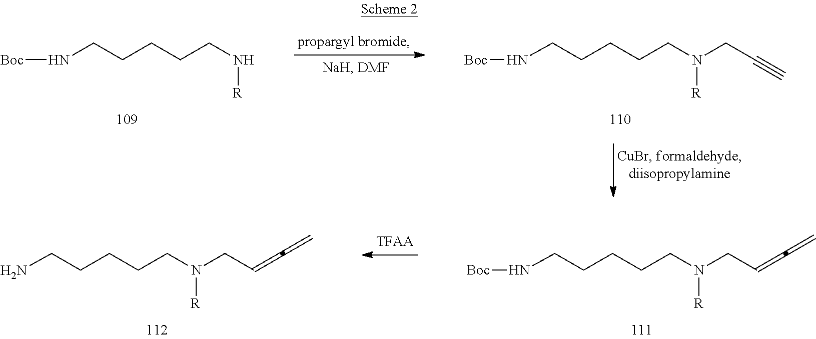

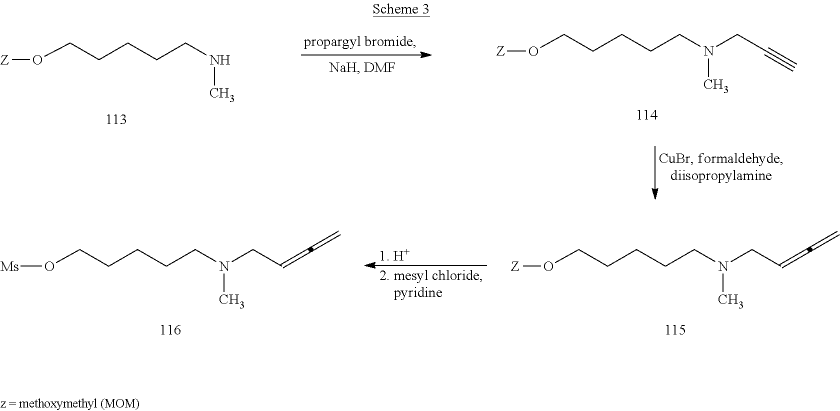

The elaboration of synthons 107, 112 and 116 to provide compounds of formula (X) and formula (XI) is outlined in Scheme 4, Scheme 5, and Scheme 6. Removal of the N-Boc protecting group in synthon 107 (Scheme 4) affords the free amine 119, which is then appended to a protected polyaminocarboxylate of general structure 120, (Casero, Jr. R. A. et al., J. Med. Chem., 44:1-26 (2001); Bellevue, F. H. et al., Bioorg. Med. Chem. Lett., 6:2765-2770 (1996); Saab, N. H. et al., J. Med. Chem., 36:2998-3004 (1993); Varghese, S. et al., J. Med. Chem., 48:6350-6365 (2005); Bi, X. et al., Bioorg. Med. Chem. Lett., 16:3229-3232 (2006)) as previously described (using DCC, HOBT, DMF; see Bellevue, Saab, and Varghese references immediately preceding) to afford 121a. The ester functionality of 121a is hydrolyzed (LiOH) (see Bellevue, Saab, and Varghese references) to produce carboxylate 121b, and this intermediate is in turn converted to substituted amide 121c (using DCC, HOBT, DMF; see Bellevue, Saab, and Varghese references). The mesitylenesulfonyl protecting groups in compounds 121a-c are then removed (Yajima, H. et al., Chem. Pharm. Bull., 26:3752-3757 (1978); Roemmele, R. C., Rappoport, H., J. Org. Chem., 53:2367-2371 (1988)) to afford potential LSD1 inhibitors of formula (X) (X═OCH₃, OH or NH—R₃, W₂═NH).

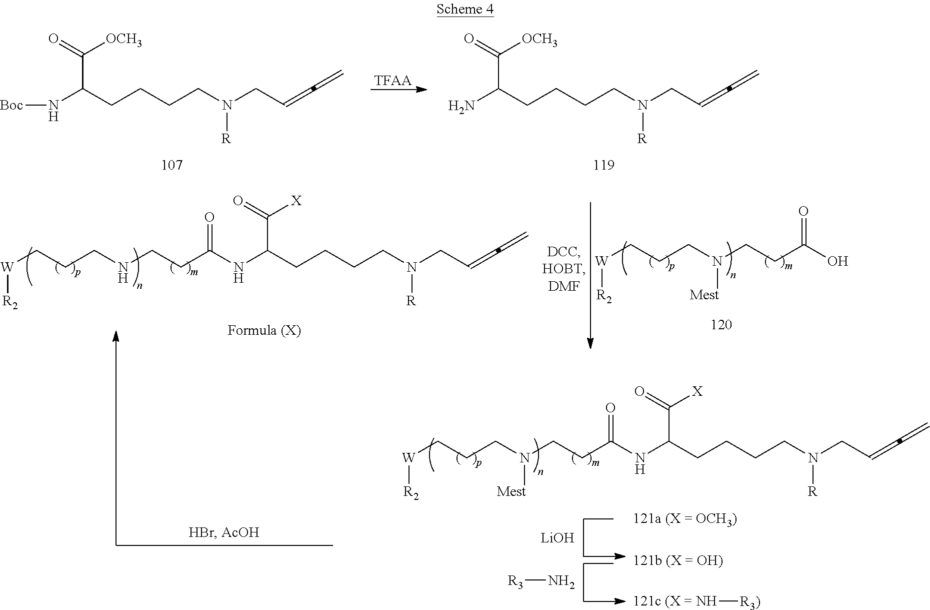

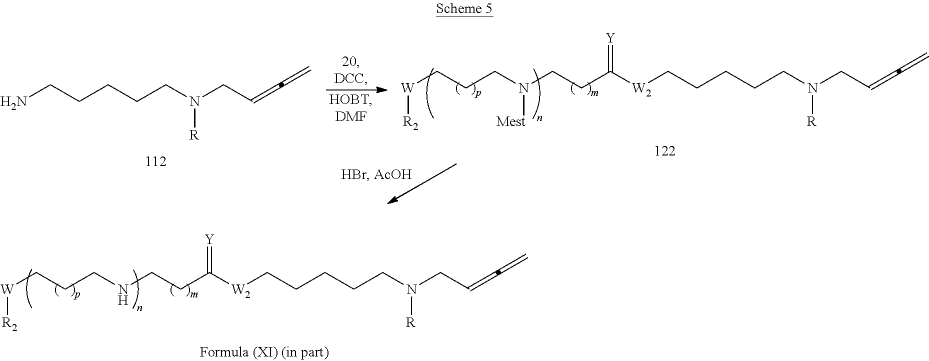

Molecules of formula (XI) can be synthesized using the route shown in Scheme 5. Synthon 112 is coupled to polyaminocarboxylate of general structure 120 (Casero, Jr. R. A. et al., J. Med. Chem., 44:1-26 (2001); Bellevue, F. H. et al., Bioorg. Med. Chem. Lett., 6:2765-2770 (1996); Saab, N. H. et al., J. Med. Chem., 36:2998-3004 (1993); Varghese, S. et al., J. Med. Chem., 48:6350-6365 (2005); Bi, X. et al., Bioorg. Med. Chem. Lett., 16:3229-3232 (2006)) as described above to yield intermediate 122. Removal of the mesityl protecting group (Yajima, H. et al., Chem. Pharm. Bull., 26:3752-3757 (1978); Roemmele, R. C., Rappoport, H., J. Org. Chem., 53:2367-2371 (1988)) then affords target molecules of formula (XI).

Synthetic routes to other compounds of formula (XI) are shown in Scheme 6 and Scheme 7. As shown in Scheme 6, intermediate 112 is reacted with aldehydes of general structure 123 (previously described in Casero, Jr. R. A. et al., J. Med. Chem., 44:1-26 (2001); Bellevue, F. H. et al., Bioorg. Med. Chem. Lett., 6:2765-2770 (1996); Saab, N. H. et al., J. Med. Chem., 36:2998-3004 (1993); Varghese, S. et al., J. Med. Chem., 48:6350-6365 (2005); Bi, X. et al., Bioorg. Med. Chem. Lett., 16:3229-3232 (2006)) using standard reductive amination conditions, followed by deprotection (30% HBr in AcOH) (Yajima, H. et al., Chem. Pharm. Bull., 26:3752-3757 (1978); Roemmele, R. C., Rappoport, H., J. Org. Chem., 53:2367-2371 (1988)) to afford compounds of formula (XI). Alternatively, synthon 116 is reacted with amines, guanidines or biguanides of general structure 124, and subsequent deprotection (30% HBr in AcOH) then affords compounds of formula (XI).

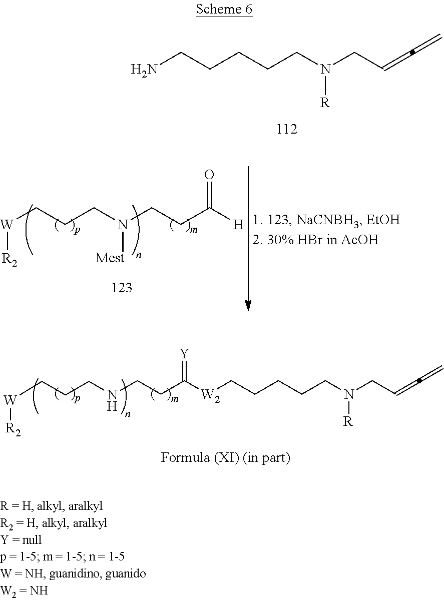

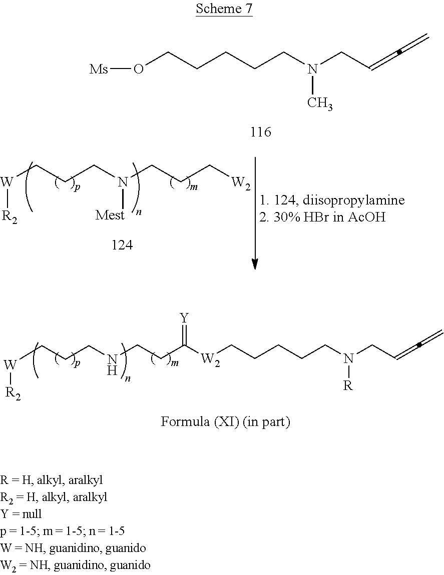

Compounds of Formula (XII) and Formula (XIII) can be synthesized using routes similar to those shown in Schemes 1-7. Intermediate 105 (Scheme 8) is alkylated (Saab, N. H. et al., J. Med. Chem., 36:2998-3004 (1993)) followed by ester hydrolysis and removal of the N-Boc group as described above to afford synthon 129. Compound 129 is then appended to a suitable polyamine precursor (Casero, Jr. R. A. et al., J. Med. Chem., 44:1-26 (2001); Bellevue, F. H. et al., Bioorg. Med. Chem. Lett., 6:2765-2770 (1996); Saab, N. H. et al., J. Med. Chem., 36:2998-3004 (1993); Varghese, S. et al., J. Med. Chem., 48:6350-6365 (2005); Bi, X. et al., Bioorg. Med. Chem. Lett., 16:3229-3232 (2006)) and elaborated as described above to yield target compounds of Formula (XII).

The chloromethyl ketone compounds of formula (XVI) are synthesized by substituting intermediate 130 for 129, as indicated in Scheme 8, followed by elaboration as described. Chloromethyl ketone derivatives act as irreversible, active site-directed inhibitors of proteases and other enzymes, and would also be expected to inactivate LSD1. Intermediates of general structure 130 are commercially available or readily synthesized (Shaw, E.; Glover, G., Arch. Bioch. Bioph., 139:298-305 (1970); Biaas, A. et al., J. Med. Chem., 49:1744-1753 (2006)), and can be coupled to the appropriate polyamine precursor via peptide coupling, as described above and previously reported (Biaas, A. et al., J. Med. Chem., 49:1744-1753 (2006)).

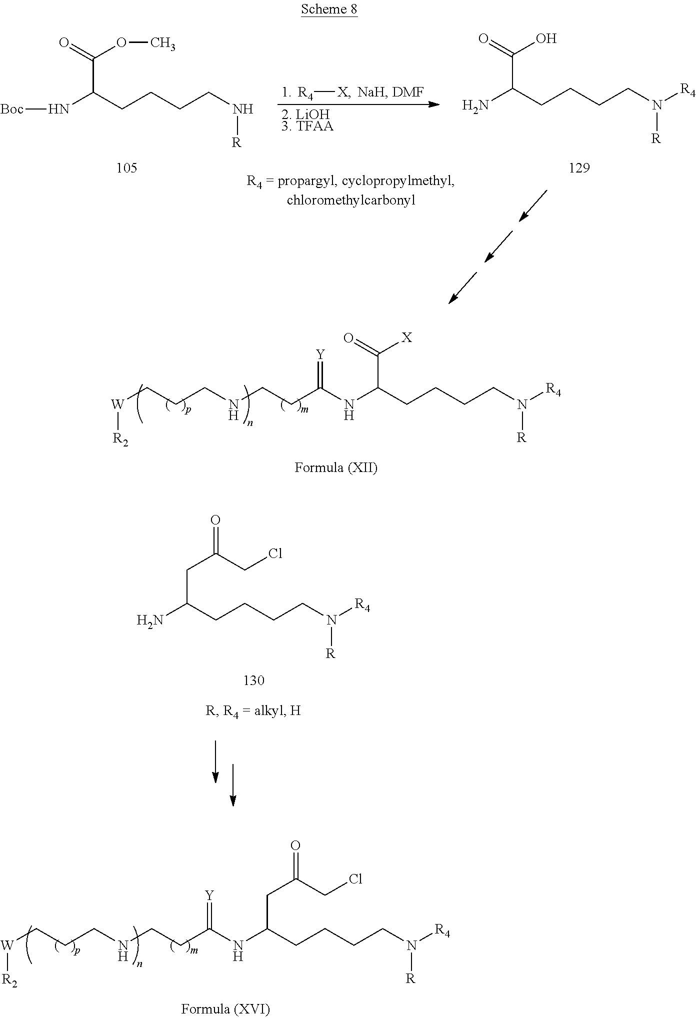

Two synthetic pathways are utilized to produce target compounds of Formula (XIII), as shown in Scheme 9. Intermediate 109 is alkylated (Saab, N. H. et al., J. Med. Chem., 36:2998-3004 (1993)) using a variety of electrophilic or latent electrophilic alkyl halides (e.g. propargyl, cyclopropylmethyl, 1-difluoroethyl, etc.), followed by removal of the N-Boc group as described above to afford synthon 132. Compound 132 is then appended to a suitable polyamine precursor (Casero, Jr. R. A. et al., J. Med. Chem., 44:1-26 (2001); Bellevue, F. H. et al., Bioorg. Med. Chem. Lett., 6:2765-2770 (1996); Saab, N. H. et al., J. Med. Chem., 36:2998-3004 (1993); Varghese, S. et al., J. Med. Chem., 48:6350-6365 (2005); Bi, X. et al., Bioorg. Med. Chem. Lett., 16:3229-3232 (2006)) and elaborated as described above to yield target compounds of formula (XIII). Alternatively, synthon 113 (Scheme 9) is alkylated (Saab, ibid.) followed by acid deprotection of the alcohol and conversion of the corresponding hydroxyl group to mesylate to afford 133. Compound 133 is then appended to a suitable polyamine precursor (Casero et al.; Bellevue et al.; Saab et al.; Varghese et al. Bi et al., ibid.) and elaborated as described above to yield target compounds of formula (XIII).

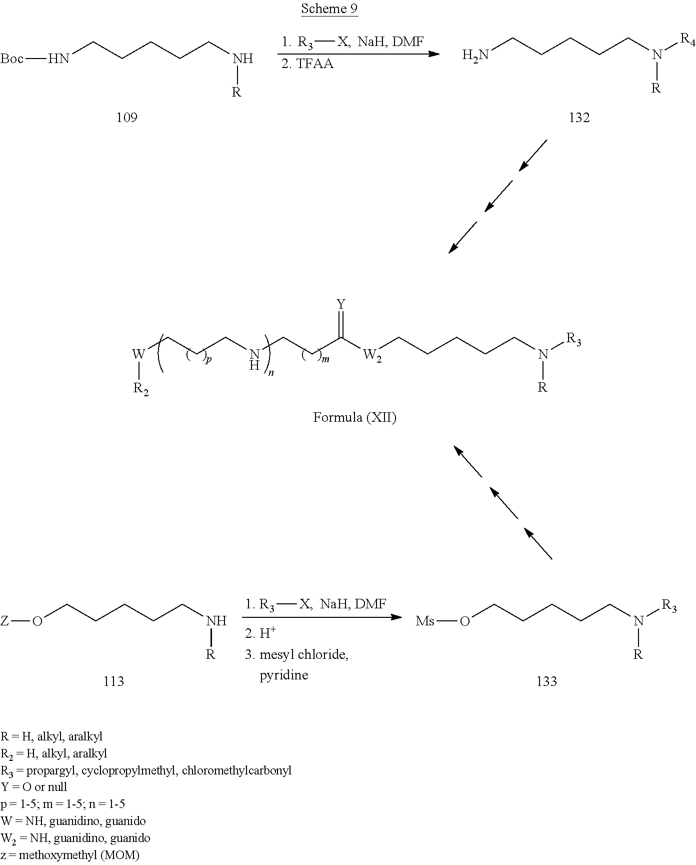

Formula (XIV) and formula (XV) contain a cyclopropylamine moiety. The synthesis of these analogues proceeds via the production of substituted cyclopropanes 120, 122 and 124, which are accessed from terminal olefins 134, 136 and 138, respectively by cyclopropanation/amination (Raju, B. et al., Bioorg. Med. Chem. Lett., 14:3103-3107 (2004)), followed by alkylation (Casero et al.; Bellevue et al.; Saab et al.; Varghese et al. Bi et al., ibid.) of the resulting terminal amine as described above. The synthesis leading to compounds of Formula (XIV) is shown in Scheme 10. The commercially available olefin 134 undergoes cyclopropanation using the method of Borne et al. (N₂CHCO₂C₂H₅ followed by ethyl chloroformate and sodium azide) (Raju, B. et al., Bioorg. Med. Chem. Lett., 14:3103-3107 (2004)), after which alkyl substituents are added to the nitrogen using reductive amination and then nucleophilic substitution, as previously described to afford 135 (Casero et al.; Bellevue et al.; Saab et al.; Varghese et al. Bi et al., ibid.). Synthon 135 is then elaborated as described above to yield compounds of Formula (XIV).

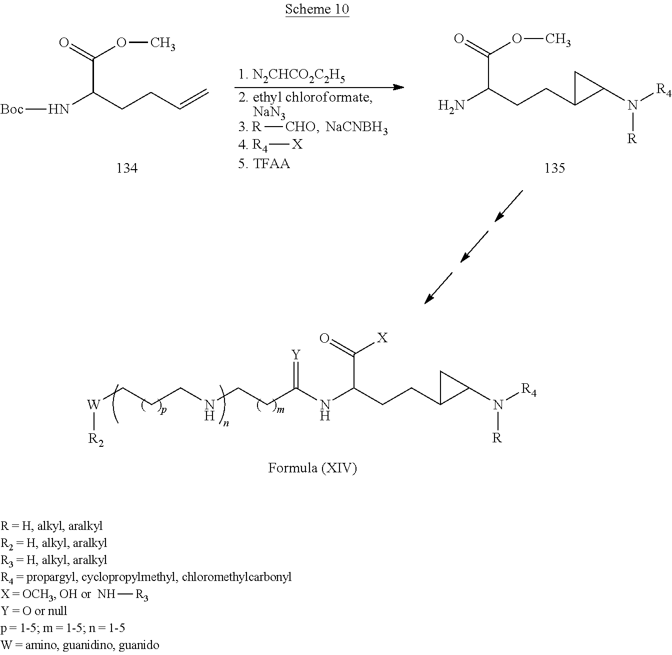

A similar approach can be used to produce compounds of formula (XV), as shown in Scheme 11. The commercially available olefin 136 is converted to the corresponding cyclopropylamine as described above, followed by sequential addition of the N-alkyl groups by reductive amination and nucleophilic substitution (see above). Removal of the N-Boc protecting group then affords 137, which is elaborated as described to afford compounds of formula (XV). Olefin 138 is likewise converted to the cyclopropylamine derivative and bis-alkylated, after which acid-catalyzed removal of the MOM protecting group (indicated by Z in Scheme 11 and other schemes) and subsequent mesylation (Saab, N. H. et al., J. Med. Chem., 36:2998-3004 (1993)) yields the desired synthon 139. Elaboration as described above then affords compounds of formula (XV).

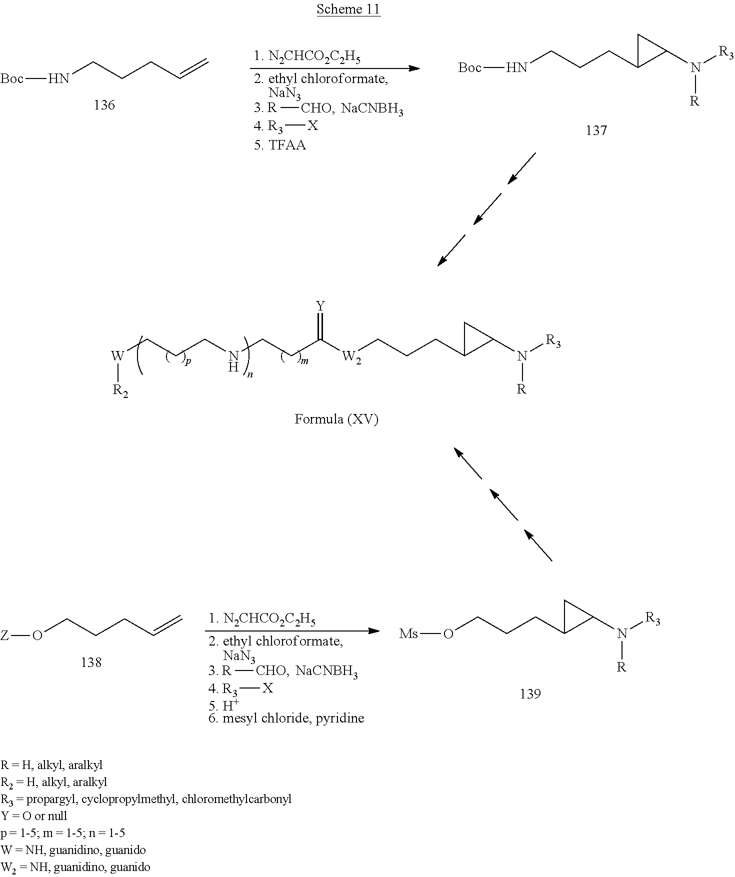

### Biological Applications—Lysine-Specific Demethylase-1 (LSD1) Inhibitors

Histones are proteins found in eukaryotic cells which act as support scaffolds for DNA (sometimes compared to a protein spool supporting the DNA thread). Histones, together with other proteins and DNA, form the chromatin of the cell nucleus. Because of their close association with DNA, histones play a role in gene regulation. The tails of histone proteins are a frequent site for covalent modifications which affect gene expression.

The enzyme lysine-specific demethylase-1 (LSD1; also known as lysine-specific histone demethylase, BHC110 and KIAA0601) is an enzyme that affects the covalent modification of histone tails, by demethylating lysine 4 of the histone H3. Shi et al. (Cell, 119:941 (2004)) showed that RNAi inhibition of LSD1 led to an increase in H3 lysine 4 methylation, followed by de-repression of the target genes. Thus LSD1 apparently represses transcription by demethylating histone H3. Conversely, inhibition of LSD1 allows transcription by preventing demethylation.

Because of the observed homology between the active site of LSD1 and monoamine oxidase (MAO), Lee et al. (Chemistry & Biology 13:563 (2006)) tested various MAO inhibitors for their ability to inhibit LSD1. They identified tranylcypromine ((1R,2S)-2-phenylcyclopropan-1-amine) as an inhibitor with an IC₅₀ less than 2 micromolar. Treating P19 embryonal carcinoma cells with tranylcypromine led to transcriptional de-repression of the Egr1 and Oct4 genes.

International Patent Application No. WO 2006/071608 is directed to a method for monitoring eukaryotic histone demethylase activity, methods for up-regulating and down-regulating methylated histone-activated genes, and a method for treating or preventing a disease (e.g., a hyperproliferative disease such as cancer) by modulating the level of protein or the activity of a histone demethylase. In view of the importance of gene regulation, and the ability to affect gene regulation by inhibiting or modulating LSD1, inhibitors of the enzyme may have significant therapeutic potential; Bi, X. et al., Bioorg. Med. Chem. Lett. 16:3229-3232 (2006) and International Patent Application No. WO 2007/021839 describes certain compounds useful as inhibitors of LSD1.

Lysine-specific demethylase-1-inhibiting compounds of the current inventions can inhibit LSD1 by at least about 25%, at a concentration of the compound of about 10 micromolar or less, about 1 micromolar or less, about 100 nanomolar or less, about 10 nanomolar or less, or about 1 nanomolar or less; by at least about 50%, at a concentration of the compound of about 10 micromolar or less, about 1 micromolar or less, about 100 nanomolar or less, about 10 nanomolar or less, or about 1 nanomolar or less; at least about 75%, at a concentration of the compound of about 10 micromolar or less, about 1 micromolar or less, about 100 nanomolar or less, about 10 nanomolar or less, or about 1 nanomolar or less; at least about 90%, at a concentration of the compound of about 10 micromolar or less, about 1 micromolar or less, about 100 nanomolar or less, about 10 nanomolar or less, or about 1 nanomolar or less; at least about 95%, at a concentration of the compound of about 10 micromolar or less, about 1 micromolar or less, about 100 nanomolar or less, about 10 nanomolar or less, or about 1 nanomolar or less; or at least about 99% at a concentration of the compound of about 10 micromolar or less, about 1 micromolar or less, about 100 nanomolar or less, about 10 nanomolar or less, or about 1 nanomolar or less.

### Biological Applications—Treatment of Cancer

“Treating” or “to treat” a disease using the methods of the invention is defined as administering one or more polyamines or polyamine analogs, with or without additional therapeutic agents, in order to palliate, ameliorate, stabilize, reverse, slow, delay, prevent, reduce, or eliminate either the disease or the symptoms of the disease, or to retard or stop the progression of the disease or of symptoms of the disease. “Therapeutic use” of the polyamines and polyamine analogs is defined as using one or more polyamines or polyamine analogs to treat a disease (including to prevent a disease), as defined above. A “therapeutically effective amount” is an amount sufficient to treat (including to prevent) a disease, as defined above. Prevention or suppression can be partial or total.

The compounds disclosed herein have anticancer activity, which has been demonstrated in a variety of human tumor cell types representing the major forms of lung, breast, prostate, and colon cancers. Thus the compounds disclosed herein can be used to treat cancer, including lung cancer (including, but not limited to, small cell lung cancer or SCLC, non-small cell lung cancer or NSCLC, alveolar epithelial cell cancer, bronchial epithelial cell cancer, and squamous cell carcinoma), breast cancer, prostate cancer, and colon cancer, or to prevent cancer, including prevention of lung cancer (including, but not limited to, small cell lung cancer or SCLC, non-small cell lung cancer or NSCLC, alveolar epithelial cell cancer, bronchial epithelial cell cancer, and squamous cell carcinoma), breast cancer, prostate cancer, and colon cancer.

### EXAMPLES

### Example 1

### Testing of Compounds for Inhibition of LSD1

Compounds of the invention are tested for inhibitory activity against lysine-specific demethylase-1 as outlined in Bi, X. et al., Bioorg. Med. Chem. Lett. 16:3229-3232 (2006) (see also Supplementary Data).

### Example 2

### In Vitro Testing of Compounds Against Cancer Cell Lines

MTS dose response experiments in H157, H82, A549, and/or Beas2B cells cells following a 96 hr exposure with compounds of the invention are performed. MTS is a standard colorimetric assay used for measuring metabolic activity in cells. MTS experiments are performed by CellTiter 96® AQᵤₑᵤₒₛ One Solution Cell Proliferation Assay from Promega Corporation. Cells are seeded at 3000 cells/well on a 96 well tissue culture plate containing 100 ul of medium/well and are allowed to attach overnight. The medium is aspirated and replaced with 100 ul of fresh medium containing the appropriate concentration of the compound being tested; the cells are then incubated for 96 hrs at 37° C. and 5% CO₂. Compounds are tested at concentrations ranging from 0.1 micromolar to 50 micromolar. Wells not containing the test compound are used as a control. Following treatment, 20 ul of MTS reagent is added to each well and incubated at 37° C. for 1.5 hrs. The absorbance of each well is then measured at 490 nm and used to determine the metabolic activity of the cells in the presence of the test compound, relative to the control. IC₅₀ values for the test compounds are extracted based on the results.

MTT dose response experiments in 235, MCF7, 435, and 10A cells following exposure to compounds of the invention are performed. MTT is a standard colorimetric assay used for measuring metabolic activity in cells. About 200 ul of media not containing cells are added to column A of a 96 well plate and used as a blank. About 200 ul of media containing cells are added to the remaining wells and incubated overnight. The remaining wells contain about 4000-5000 MCF7 cells/well, 3000 231 cells/wells, 12,000 468 cells/well, or 9000 MCF 10A cells/well. Following incubation, the media in the wells is aspirated and replaced with 200 ul of fresh media in columns A and B of the 96 well plate. Column B is used as a control. Next 200 ul of fresh media containing the compound being tested is added to the remaining wells and incubated for 96 hrs. Compounds are routinely tested at concentrations ranging from 0.1 micromolar to 50 micromolar. Following incubation for 96 hrs, the media in each well is aspirated and replaced with 100 ul of 5 mg/ml MTT (3-(4,5-Dimethylthiazol-2-yl)-2,5-diphenyltetrazolium bromide) solution in Serum-Free media and incubated for 4 hours. Following incubation with MIT solution, the MIT solution is removed from the wells and replaced with 200 ul of a 1:1 EtOH+DMSO solution and incubated for 20 minutes. Following incubation with the EtOH+DMSO solution the plates are read at 540 nm and are used to determine the metabolic activity of the cells in the presence of the test compound, relative to the control. IC₅₀ values for the test compounds are extracted based on the results.

SSAT (spermidine/spermine-N¹-acetyltransferase) activity experiments in H157, H82, and A549 cells following exposure to compounds of the invention are performed. A detailed protocol for determining SSAT activity is described in Casero et al., Cancer Research, 49:3829 (1989). Briefly, the SSAT activity is measured by harvesting the treated cells at the exposure time. The cells are then lysed and treated with spermidine, and 1-[¹⁴C]acetyl coenzyme A for 5 minutes. Enzyme activity is measured in term of picomoles of [¹⁴C]acetylspermidine formed per mg of cell protein per min (pmol/mgP/min).

Experiments to measure putrescine, spermidine, and spermine polyamine levels in H157 and H82 cells following exposure to compounds of the invention are performed. Polyamine levels are determined using the precolumn dansylation labeling, reverse-phase high-pressure liquid chromatography method as described by Kabra et al., J. Chromotography, 380:19 (1986).

SMO (Spermine Oxidase) activity in H157 cells following exposure to compounds of the invention is performed. A detailed protocol for measuring SMO activity is described in Wang et al., Cancer Research, 61:5370 (2001).

ODC (Ornithine decarboxylase) activity experiments in H157 following exposure to compounds of the invention are performed. A detailed protocol for measuring ODC activity is described in Pegg et al., Methods Enzymology, 94:158 (1983).

Treatment induced cell cycle measurements in H157 cells are performed. Following exposure of the cells to a compound of interest, at a concentration of 10 uM, for 24 hrs, the cells are harvested, prepared and transferred to a FACS for cell cycle analysis. (See Carlisle et al., Clinical Cancer Research 8:2684 (2002) and references therein.)

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A compound of the following formula (I): 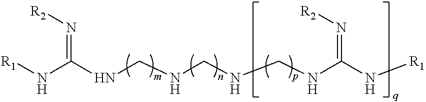
or a salt, solvate, or hydrate thereof, wherein n is an integer from 1 to 12, m and p are independently an integer from 1 to 5, q is 0 or 1, each R₁ is independently selected from the group consisting of C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, C₇-C₂₄ substituted or unsubstituted heteroaralkyl or C₁-C₈ alkyl-CH═C═CH₂ or C₁-C₈ alkyl-C≡CH or C₁-C₈ alkyl-cyclopropane or C(═O)C₁-C₈alkyl substituted with at least one halo group or cyclopropyl-NR₂ where each R is independently H, alkyl, or aralkyl, with the proviso that at least one R₁ is C₁-C₈ alkyl-CH═C═CH₂, C₁-C₈ alkyl-C≡CH, C₁-C₈ alkyl-cyclopropane, or C(═O)C₁-C₈alkyl substituted with at least one halo group or cyclopropyl-NR₂ where each R is independently H, alkyl, or aralkyl; and each R₂ is independently selected from hydrogen or a C₁-C₈ substituted or unsubstituted alkyl.

2. A compound of the following formula (II): 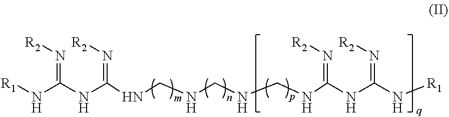
or a salt, solvate, or hydrate thereof, wherein n is an integer from 1 to 12, m and p are independently an integer from 1 to 5, q is 0 or 1, each R₁ is independently selected from the group consisting of C₁-C₈ substituted or unsubstituted alkyl, C₆-C₂₀ substituted or unsubstituted aryl, C₆-C₂₀ substituted or unsubstituted heteroaryl, C₇-C₂₄ substituted or unsubstituted aralkyl, C₇-C₂₄ substituted or unsubstituted heteroaralkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one R₁ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl.

3. A compound of the following formula (III): 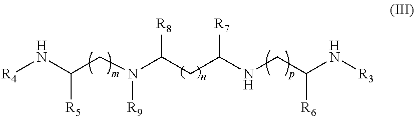
or a salt, solvate, or hydrate thereof, wherein n is an integer from 1 to 12; m and p are independently an integer from 1 to 5; R₃ and R₄ are independently selected from the group consisting of hydrogen, C₁-C₈ substituted or unsubstituted alkyl, C₆-C₂₀ substituted or unsubstituted aryl, C₇-C₂₄ substituted or unsubstituted aralkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₃ and R₄ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; each R₂ is independently hydrogen or a C₁-C₈ substituted or unsubstituted alkyl; R₅, R₉, R₆, R₇ and R₈ are independently selected from the group consisting of hydrogen and C₁-C₈ substituted or unsubstituted alkyl; and wherein either m and p are not the same integer or at least one of R₅, R₉, R₆, R₇ and R₈ is a C₁-C₈ substituted or unsubstituted alkyl.

4. A compound of the following formula (IV): 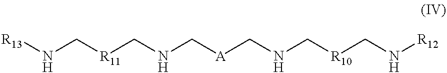
or a salt, solvate, or hydrate thereof, wherein A, R₁₀ and R₁₁ are independently (CH₂)ₙ or ethene-1,1-diyl; n is an integer from 1 to 5; R₁₂ and R₁₃ are independently selected from the group consisting of hydrogen, C₂-C₈ substituted or unsubstituted alkenyl, C₁-C₈ substituted or unsubstituted alkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₁₂ and R₁₃ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl.

5. A compound of the following formula (V): 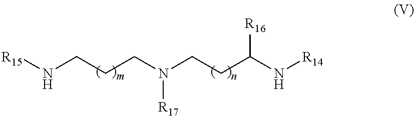
or a salt, solvate, or hydrate thereof, wherein n is an integer from 1 to 8; m is an integer from 1 to 8; R₁₅ and R₁₄ are independently selected from the group consisting of hydrogen, C₁-C₈ substituted or unsubstituted n-alkyl or (C₃-C₈) branched alkyl, C₆-C₂₀ substituted or unsubstituted aryl or heteroaryl, C₇-C₂₄ substituted or unsubstituted aralkyl or heteroaralkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₁₅ and R₁₄ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl; R₁₆ and R₁₇ are independently hydrogen or a C₁-C₈ substituted or unsubstituted alkyl; and wherein the compound contains no more than three secondary amino groups except when R₁₇ is a C₁-C₈ substituted or unsubstituted alkyl and wherein the compound is free from a methylphosphonate or hydroxy moiety.

6. A compound of the following formula (VI): 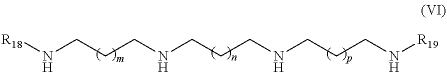
or a salt, solvate, or hydrate thereof, wherein n is an integer from 1 to 12; m and p are independently an integer from 1 to 5; R₁₈ and R₁₉ are independently selected from the group consisting of hydrogen, C₁-C₈ unsubstituted alkyl (e.g., methyl, ethyl, tert-butyl, isopropyl, pentyl, cyclobutyl), C₁-C₈ n-alkyl substituted with a cycloalkyl group comprising at least two rings, C₇-C₂₄ substituted or unsubstituted aralkyl or heteroaralkyl comprising at least two rings, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₁₈ and R₁₉ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl.

7. A compound of the following formula (VII): 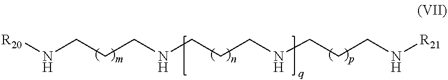
or a salt, solvate, or hydrate thereof, wherein n is an integer from 1 to 12; m and p are independently an integer from 1 to 5; q is 0 or 1; R₂₀ and R₂₁ are independently selected from the group consisting of hydrogen, C₁-C₈ substituted or unsubstituted alkyl, —C(═O)—C₁-C₈ substituted or unsubstituted alkyl, —C(═O)—C₁-C₈ substituted or unsubstituted alkenyl, —C(═O)—C₁-C₈ substituted or unsubstituted alkynyl, C₇-C₂₄ substituted or unsubstituted aralkyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₂₀ and R₂₁ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl.

8. A compound of the following formula (VIII): 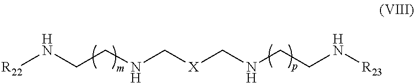
or a salt, solvate, or hydrate thereof, wherein m and p are independently an integer from 1 to 5; X is —(CH₂)n- or cyclohex-1,3-diyl; n is an integer from 1 to 5; R₂₂ and R₂₃ are independently selected from the group consisting of hydrogen, n-butyl, ethyl, cyclohexylmethyl, cyclopentylmethyl, cyclopropylmethyl, cycloheptylmethyl, cyclohexyleth-2-yl, benzyl, C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl, with the proviso that at least one of R₂₂ and R₂₃ is C₁-C₈ alkyl-CH═C═CH₂ or C₁-C₈ alkyl-C≡CH or C₁-C₈ alkyl-cyclopropane or C(═O)C₁-C₈ alkyl substituted with at least one halo group or cyclopropyl-NR₂ where each R is independently H, alkyl, or aralkyl; and each R₂ is independently hydrogen or a C₁-C₈ substituted or unsubstituted alkyl.

9. A compound of the following formula (IX): 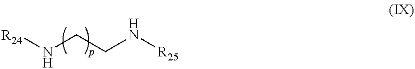
or a salt, solvate, or hydrate thereof, wherein p is an integer from 1 to 5; R₂₄ is an amino-substituted cycloalkyl (e.g., a cycloalkyl group substituted with a primary, secondary, tertiary or quaternary amine), a C₂-C₈ substituted or unsubstituted alkanoyl (which substituted alkanoyl may be substituted with one or more substituents such as those listed for “Substituted alkyl” including without limitation an alkanoyl substituted with a methyl and an alkylazide group), C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl; and R₂₅ is a C₁-C₈ substituted or unsubstituted alkyl, a C₇-C₂₄ substituted or unsubstituted aralky (I)-(VIII), C₁-C₈ alkyl-CH═C═CH₂, C₁-C₈ alkyl-C≡CH, C₁-C₈ alkyl-cyclopropane, C(═O)C₁-C₈alkyl substituted with at least one halo group and cyclopropyl-NR₂ where each R is independently H, alkyl, or aralkyl with the proviso that at least one of R₂₄ and R₂₅ is C₁-C₈ alkyl-CH═C═CH₂ or C₁-C₈ alkyl-C≡CH or C₁-C₈ alkyl-cyclopropane or C(═O)C₁-C₈alkyl substituted with at least one halo group or cyclopropyl-NR₂ where each R is independently H, alkyl, or aralkyl; and each R₂ is independently hydrogen or a C₁-C₈ substituted or unsubstituted alkyl.

10. A compound of the following formula (X): 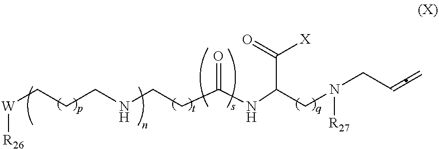
or a salt, solvate, or hydrate thereof, wherein R₂₆ is hydrogen, C₁-C₈alkyl or aralkyl where the alkyl moiety of the aryl group is a C₁-C₈alkyl, W is —NH—, 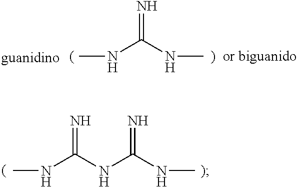
p and n are independently an integer from 1 to 5; t is an integer from 1 to 6; q is an integer from 1 to 10; s is 0 or 1; X is —O—C₁-C₈alkyl, OH or NHR₂₈, where R₂₈ is hydrogen, C₁-C₈alkyl or aralkyl where the alkyl moiety of the aryl group is a C₁-C₈alkyl; R₂₇ is hydrogen, C₁-C₈alkyl or aralkyl where the alkyl moiety of the aryl group is a C₁-C₈alkyl.

11. A compound of the following formula (XI): 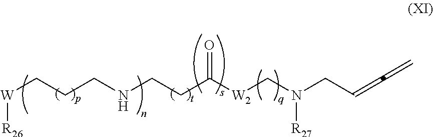
or a salt, solvate, or hydrate thereof, wherein R₂₆ is hydrogen, C₁-C₈alkyl or aralkyl where the alkyl moiety of the aryl group is a C₁-C₈alkyl, W is —NH—, 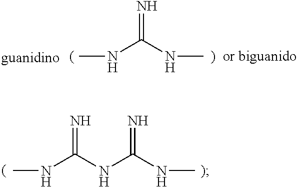
p and n are independently an integer from 1 to 5; t is an integer from 1 to 6; q is an integer from 1 to 10; s is 0 or 1; R₂₇ is hydrogen, C₁-C₈alkyl or aralkyl where the alkyl moiety of the aryl group is a C₁-C₈alkyl; and W₂ is —NH—,

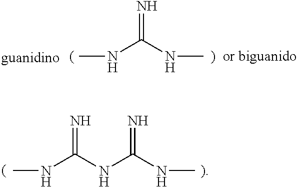

12. A compound of the following formula (XII): 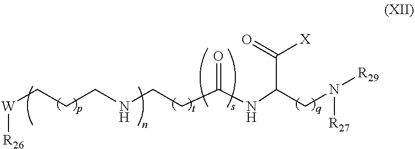
or a salt, solvate, or hydrate thereof,
wherein R₂₆ is hydrogen, C₁-C₈alkyl or aralkyl where the alkyl moiety of the aryl group is a C₁-C₈alkyl; W is —NH—, 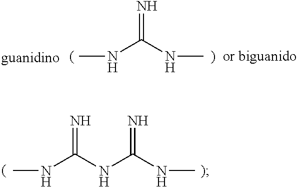
p and n are independently an integer from 1 to 5; t is an integer from 1 to 6; q is an integer from 1 to 10; s is 0 or 1; R₂₇ is hydrogen, C₁-C₈alkyl or aralkyl where the alkyl moiety of the aryl group is a C₁-C₈alkyl; and R₂₉ is C₁-C₈alkyl-C≡CH, C₁-C₈cyclopropane or C(═O)C₁-C₈alkyl substituted with at least one halo group.

13. A compound of the following formula (XIII): 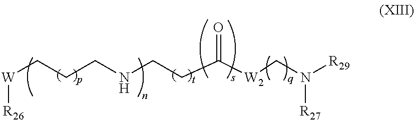
or a salt, solvate, or hydrate thereof,
wherein R₂₆ is hydrogen, C₁-C₈alkyl or aralkyl where the alkyl moiety of the aryl group is a C₁-C₈alkyl;
W is —NH—, 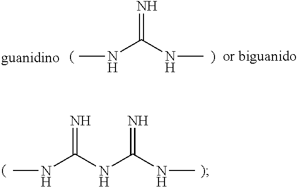
p and n are independently an integer from 1 to 5; t is an integer from 1 to 6; q is an integer from 1 to 10; s is 0 or 1; R₂₇ is hydrogen, C₁-C₈alkyl or aralkyl where the alkyl moiety of the aryl group is a C₁-C₈alkyl; and R₂₉ is C₁-C₈ alkyl-C≡CH, C₁-C₈cyclopropane or C(═O)C₁-C₈alkyl substituted with at least one halo group; and W₂ is —NH—,

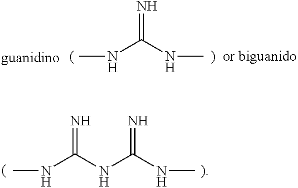

14. A compound of the following formula (XIV): 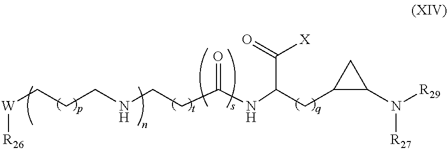
or a salt, solvate, or hydrate thereof,
wherein R₂₆ is hydrogen, C₁-C₈alkyl or aralkyl where the alkyl moiety of the aryl group is a C₁-C₈alkyl;
W is —NH—, 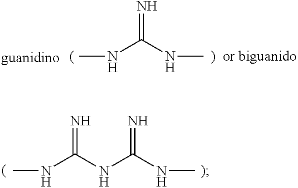
p and n are independently an integer from 1 to 5; t is an integer from 1 to 6; q is an integer from 1 to 10; s is 0 or 1; R₂₇ is hydrogen, C₁-C₈alkyl or aralkyl where the alkyl moiety of the aryl group is a C₁-C₈alkyl; and R₂₉ is C₁-C₈ alkyl-C≡CH, C₁-C₈cyclopropane or C(═O)C₁-C₈alkyl substituted with at least one halo group.

15. A compound of the following formula (XV): 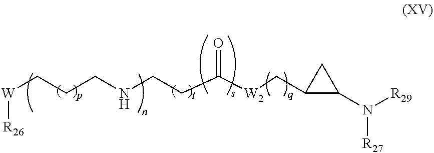
or a salt, solvate, or hydrate thereof,
wherein R₂₆ is hydrogen, C₁-C₈alkyl or aralkyl where the alkyl moiety of the aryl group is a C₁-C₈alkyl;
W is —NH—, 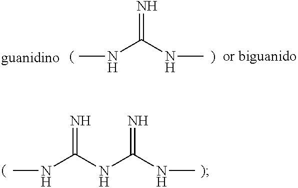
p and n are independently an integer from 1 to 5; t is an integer from 1 to 6; q is an integer from 1 to 10; s is 0 or 1; R₂₇ is hydrogen, C₁-C₈alkyl or aralkyl where the alkyl moiety of the aryl group is a C₁-C₈alkyl; and R₂₉ is C₁-C₈alkyl-C≡CH, C₁-C₈cyclopropane or C(═O)C₁-C₈alkyl substituted with at least one halo group.

16. A compound of the following formula (XVI): 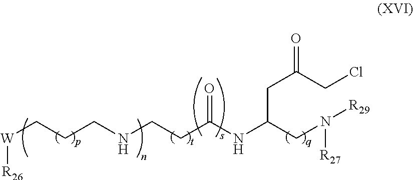
or a salt, solvate, or hydrate thereof,
wherein R₂₆ is hydrogen, C₁-C₈alkyl or aralkyl where the alkyl moiety of the aryl group is a C₁-C₈alkyl, W is —NH—, 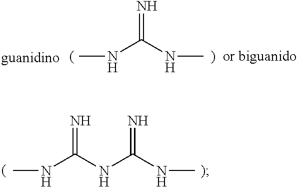
p and n are independently an integer from 1 to 5; t is an integer from 1 to 6; q is an integer from 1 to 10; s is 0 or 1; X is —O—C₁-C₈alkyl, OH or NHR₂₈, where R₂₈ is hydrogen, C₁-C₈alkyl or aralkyl where the alkyl moiety of the aryl group is a C₁-C₈alkyl; R₂₇ is hydrogen, C₁-C₈alkyl or aralkyl where the alkyl moiety of the aryl group is a C₁-C₈alkyl; and R₂₉ is C₁-C₈alkyl-C≡CH, C₁-C₈cyclopropane or C(═O)C₁-C₈alkyl substituted with at least one halo group.

17. A pharmaceutical composition comprising a compound of claim 1 and a pharmaceutically acceptable carrier.

18. A kit comprising a compound of claim 1 and instructions for therapeutic use of the compound.

19. (canceled)

20. A method of treating cancer in a subject, comprising administering to the subject a compound of claim 1, or any salt, solvate, hydrate or stereoisomer thereof.

21. The method of claim 20 wherein the subject is identified as suffering from cancer and the compound is administered to the identified subject.

22-25. (canceled)
